(19) United States
(12) Patent Application Publication
(10) Pub. No.: US 2013/0302798 A1
(43) Pub. Date: Nov. 14, 2013

(54) DETECTION OF MUTATIONS, IN PARTICULAR DELETIONS OR INSERTIONS

(71) Applicants: Martin Schuler, Essen (DE); Frank Breitenbuecher, Muelheim (DE); Sandra Hoffarth, Muelheim (DE); Sarah-Luise Boeck, Herten (DE)

(72) Inventors: Martin Schuler, Essen (DE); Frank Breitenbuecher, Muelheim (DE); Sandra Hoffarth, Muelheim (DE); Sarah-Luise Boeck, Herten (DE)

(73) Assignee: UNIVERSITAET DUISBURG-ESSEN, ESSEN (DE)

(21) Appl. No.: 13/808,600

(22) PCT Filed: Jul. 4, 2011
(86) PCT No.: PCT/EP2011/003297

§ 371 Date: Jul. 30, 2013

(30) Foreign Application Priority Data
Jul. 9, 2010 (DE) 10 2010 026 736.8
Dec. 11, 2010 (DE) 10 2010 054 193.1

(51) Int. Cl. C12Q 1/68

(52) U.S. Cl. CPC C12Q 1/6886
USPC 435/6.11

(57) ABSTRACT

A method for detecting at least one gene modification, such as a mutation in a gene, such as a gene that codes for a protein associated with at least one of a tumor and a cancer. The method includes providing a detectable hybridization probe (sensor probe) which interacts with/binds to a gene not having a gene modification (wild type gene) and with a gene having a gene modification (mutation gene). The detectable hybridization probe (sensor probe) has at least one of a higher specificity, a higher binding affinity and a higher selectivity for the gene not having a gene modification (wild type gene) compared to the gene having a gene modification (mutation gene). At least one gene modification is detected with the detectable hybridization probe (sensor probe).

### CROSS REFERENCE TO PRIOR APPLICATIONS

This application is a U.S. National Phase application under 35 U.S.C. §371 of International Application No. PCT/EP2011/003297, filed on Jul. 4, 2011 and which claims benefit to German Patent Application No. 10 2010 026 736.8, filed on Jul. 9, 2010, and to German Patent Application No. 10 2010 054 193.1, filed Dec. 11, 2010. The International Application was published in German on Jan. 12, 2012 as WO 2012/003950 A1 under PCT Article 21(2).

### FIELD

The present invention relates to the area of the detection of gene modifications, especially mutations, in genomic DNA, wherein the gene modification or mutation can in particular be associated with a tumor and/or cancer disease, such as a bronchial carcinoma. Based on detection of the gene modification, therapeutic approaches or measures can be suitably optimized for targeted treatment of the cancer or tumor disease. As described below, the gene modification is in particular a frameshift mutation, for example, a deletion or insertion.

The present invention relates in particular to a method of detecting at least one gene modification, in particular, a mutation, in a gene, for example, in a gene coding for a protein associated with a tumor and/or cancer disease.

The present invention also relates to a composition, in particular, for use in the context of an asymmetric polymerase chain reaction, which has specific components, wherein the composition according to the present invention can be ready for use, for example, in the form of an aqueous solution or dispersion or else in the form of spatially separated components based on a kit or kit-of-parts.

The present invention also relates to the use of the composition according to the present invention for detecting at least one gene modification, in particular, a mutation.

### SEQUENCE LISTING

The Sequence Listing associated with this application is filed in electronic form via EFS-Web and is hereby incorporated by reference into this specification in its entirety. The name of the text file containing the Sequence Listing is Sequence_Listing_{—}07_JAN_{—}2013. The size of the text file is 12,875 Bytes, and the text file was created on Jan. 7, 2013.

### BACKGROUND

Cancer or tumor diseases are, after diseases of the cardiovascular system, the second commonest cause of death in Germany. Not every cancer is fatal if therapy is begun early or the tumor disease does not develop until later in life and perhaps only progresses slowly. The current cure rate for all cancers is on average 30 to 40%, although there may be large differences depending on the actual cancer disease. For example, cancer diseases of the respiratory tract, especially of the lung, count as poorly treatable cancers.

In cancer diseases or in tumor cells, the coordination of growth, division and destruction or apoptosis is disturbed or disabled in the cell cluster. Endogenous regulatory signals are often not recognized or are not executed or are executed incorrectly, which is often linked causally to genetic defects or gene modifications, such as mutations. Genetic changes, such as mutations, can thus lead to changes in the structure and in the physiology of proteins encoded by the affected genes, which can induce or promote tumor growth. The development of cancer or carcinogenesis, especially the primary disease event, may thus be due to changes of the genetic material which are not compensated by endogenous monitoring and correcting systems and consequently, for example, in the context of cell division processes, can be transferred to subsequent cells, which sometimes leads to the development of a primary tumor.

Lung carcinomas, which are also designated synonymously as bronchial carcinomas, bronchogenic carcinomas or lung cancer, constitute a malignant tumor disease based on degenerated cells, especially of the bronchi or of the bronchioli. Bronchial or lung carcinoma is one of the commonest malignant human cancers and represents one of the commonest causes of death through cancer in the western hemisphere. The number of new lung cancer cases in Germany is about 50,000 persons per year. The main cause of lung cancers is smoking with inhaling. In addition, some toxic substances, such as asbestos or chromium, can also induce lung carcinomas. As symptoms are sometimes totally absent or only nonspecific in the early phases of the disease, the first diagnosis of a lung carcinoma is not generally made until the later stages of the disease so that one of the most promising therapeutic options, complete removal of the tumor by surgery, often is no longer feasible, because metastasis is also already in progress. The cure rate for bronchial carcinoma is generally very poor, with a five-year survival rate of less than 10%; the probability of survival after two years is under 20%.

About a quarter of all malignant tumors are bronchial carcinomas. In men, bronchial carcinoma is the commonest tumor disease worldwide; in Germany it is the third-commonest after prostate cancer and colorectal carcinoma, nevertheless bronchial carcinoma takes first place as cause of death from cancer.

Based on their histology and the course of the disease, lung carcinomas are generally divided into two groups, namely small cell lung cancer (SCLC) on the one hand and non-small cell lung cancer (NSCLC) on the other hand. Non-small cell lung cancer or NSCLC, with an incidence of 85% of lung cancers, represents the largest group of bronchial carcinomas. Depending on the histological findings, non-small cell carcinoma or NSCLC can be differentiated into a squamous-cell carcinoma, sometimes with a spindle-cell form, an adenocarcinoma and a large-cell carcinoma or giant-cell carcinoma.

Therapeutic approaches known in the prior art for the treatment of lung carcinoma, in particular, small cell lung cancer, focus decisively on a therapeutic approach based on chemotherapy or radiotherapy. However, such treatments have severe side effects and often do not lead to the desired therapeutic success. In studies, platinum-based combination therapies also only achieve an average extension of survival by ten to twelve months. Recently, patients with a diagnosed small cell lung cancer or NSCLC have been provided with alternatives to the usual treatment with chemotherapeutics. Medications are thus used which, in contrast to cytostatics, act specifically on tumor cells, and consequently also have far fewer side effects. These, in particular, include substances obtainable under the international nonproprietary names gefitinib, erlotinib and cetuximab, which specifically bind to or inactivate the receptor of the proliferation factor EGF (Epidermal Growth Factor), namely, the so-called EGF receptor or EGFR, which is often involved in lung cancers.

The EGF receptor (Epidermal Growth Factor receptor) is a member of the ErbB family with a subfamily of four closely-related receptor tyrosine kinases. The EGF receptor is often also designated synonymously as HER1, EGFR1 or ErbB-1.

The EGF receptor is a transmembrane receptor with intrinsic tyrosine kinase activity, which occurs in all cell types. The receptor has a membrane pore and, in the cytoplasmic portion, a kinase domain with ATP binding site. The EGF receptor belongs to the receptors for growth factors.

In nonmalignant cells, after binding of its ligand (EGF), the receptor is activated by dimerization and phosphorylation and then conveys growth and survival signals into the interior of the cell. The activation of the receptor finally leads to stimulation of cell growth and prevention of apoptosis or programmed cell death. The EGF receptor supports proliferation and cell survival.

However, overexpression and certain mutations in the EGF receptor (as can sometimes be observed in tumor cells) mediate permanent or excessive activation of the receptor, which is accompanied by an undesirably high cell growth rate, excessive cell division, and therefore tumor development or tumor growth. For malignant cells, a constant supply of growth signals is advantageous as they bring about or support the accelerated proliferation and the survival of the malignant cells. Tumor cells that have overexpression or activating mutations with respect to the EGF receptor are even dependent on the permanent or excessive activation of the EGF receptor for their proliferation and for their survival. The EGF receptor is therefore up-regulated in various tumor types or occurs in mutated form, with the result that the tumor cells in question grow uncontrollably and multiply. The aforementioned active substances aim to block the oncogenic signal of the EGF receptor and thus suppress or delay tumor growth.

The EGF receptor can thus be directly linked to a tumor or cancer disease, especially a lung or bronchial carcinoma, such as small cell lung cancer, especially as the EGF receptor in its mutated form leads to uncontrolled growth and multiplication of tumor cells. Specific blocking or inactivation of the, in particular, mutated, EGF receptor can therefore lead to restriction or suppression of the growth of tumor cells.

In the context of the present invention, it is important that through appropriate inhibition of the EGF receptor, the activation of the receptor can be reduced or inhibited. A large proportion of the mutations of the EGF receptor in patients with small cell lung cancer or NSCLC are based on various deletions in exon 19 of the EGF receptor, for example, the deletion ΔE746-A750 (i.e., omission of the amino acids in position 746 (glutamic acid) to position 750 (alanine) in the amino acid sequence of the EGF receptor), and on a point mutation in exon 21, namely the L858R mutation (i.e., substitution of the amino acid leucine L in position 858 in the amino acid sequence of the EGF receptor with the amino acid arginine R). With respect to exon 19 of the EGF receptor, overall there is a large number of deletions, which have in common that they occur in the region of the amino acid position 746 or 747 in the amino acid sequence of the EGF receptor, but differ with respect to their concrete form, in particular, with respect to the concrete modification of the amino acid sequence (cf. also the synoptic representation in FIG. 1). Such mutations can accordingly also only be detected with great difficulty or with considerable technical expenditure.

Patients with a lung tumor who have one of these modifications are particularly suitable for therapy with EGF receptor inhibitors. In particular, the medicinal products or substances gefitinib and erlotinib possess high specificity of action with respect to EGF receptors that have mutations of this kind. Therapy with specific inhibitors of the EGF receptor, in particular, with respect to its mutated form, is generally well tolerated and also has a certain efficacy. Owing to the high specificity, the receptors bearing the mutation are inhibited selectively, which reduces side effects and increases the therapeutic effect.

After a certain time, most patients develop a secondary mutation, which occurs in addition to the mutation already present and sometimes leads to resistance to erlotinib and gefitinib. In about 65% of these cases there is a mutation in exon 20 of the EGF receptor, namely a T790M mutation (i.e., substitution of the amino acid threonine T with methionine M in position 790 of the EGF receptor). For these patients, medicinal products are available whose mechanism of action and specificity differ from the first-generation medicinal products, such as erlotinib and gefitinib. The second-generation inhibitors, in particular, bind irreversibly to the receptor, and not reversibly, as is the case with the first-generation drugs in question. Patients with a small cell lung cancer, who because of the secondary mutation, especially the T790M mutation, no longer respond to first-generation drugs, can therefore be treated further with a second-generation EGF receptor inhibitor. These inhibitors are also highly specific and effective, so that the growth and survival of the tumor cells can be slowed down or prevented.

Against this technical and medical background, a mutation analysis that is rapid, simple to carry out and leads to exact results with respect to the EGF receptor in patients with a lung carcinoma is therefore tremendously important, in particular, also against the background of fine-tuning or optimizing the therapeutic procedure with respect to the specific mutation that has been found.

In particular, for ensuring an optimum therapeutic approach by means of highly effective, personalized medicine, it is necessary to investigate the tumor tissue for the status of the EGF receptor, especially with respect to any mutations that may be present, in particular, as described above. On this basis, individual patients can be treated with the corresponding EGF receptor inhibitors according to their mutation status.

Based on an informative mutation analysis with respect to the EGF receptor, targeted therapy can accordingly be carried out with the respective drugs.

Various molecular-biology methods or approaches are available in the prior art for detecting mutations in genomic DNA from tumor tissue. For example, Sanger sequencing is used as a standard. However, this method has the disadvantage that mutations can only be detected when the DNA bearing them is present at least in a proportion from 20% to 25% in the sample to be analyzed, relative to the total DNA content of the sample. Execution and evaluation moreover takes a relatively long time, as the test can take several hours.

Another method of the prior art for analysis of mutations is polymerase chain reaction (PCR), for example, real-time PCR (RT-PCR). Analysis time can be reduced by using this method. Moreover, execution is relatively inexpensive and sensitivity to the mutation to be detected or analyzed is already increased. The results obtained by conventional PCR are nevertheless not always satisfactory, especially if the sample only has extremely small amounts of mutation material. As a result, conventional PCR only has low sensitivity.

A further drawback of the methods of the prior art is that the mutations to be investigated must be known as such. In the state of the art, the fundamental principle adopted is that specific probes are used for the mutation to be investigated or to be detected in each case, with which a concrete mutation can exclusively be detected, for example, in the form of a special point mutation. The use of mutation-specific probes is not always optimal, especially with respect to detecting deletions or insertions, especially if these occur in a protein at a comparable position and therefore, as it were, as a kind of group and have different effects on the amino acid sequence.

### SUMMARY

An aspect of the present invention is to provide a method of detecting gene modifications, especially mutations, which at least partially avoids or else at least decreases the disadvantages of the prior art described above, wherein the method according to the present invention should focus as a priority on the detection of special mutations in the form of deletions or insertions.

An aspect of the present invention is to provide a method with which a large number of various kinds of gene modifications can be detected simply, while carrying out as few process steps or repetitions as possible.

An aspect of the present invention is to provide a method that has very high sensitivity, i.e., with which even very small amounts of mutation material in a sample or a material to be analyzed leads to informative results.

An aspect of the present invention is to provide a method which can be used on a large number of various kinds of samples or patient materials, for example, blood samples, lymph, cells, purified DNA or the like.

An aspect of the present invention is to provide a method which would allow a well-founded statement to be made with respect to a mutation-dependent or mutation-specific therapeutic approach for optimizing the underlying treatment regimen, especially with respect to the selection of special drugs.

An aspect of the present invention is to provide a method which is suitable for the detection or analysis of mutations in proteins, especially the EGF receptor, wherein the mutation or the protein with the mutation is associated with the development or the occurrence of lung carcinomas, especially of small cell lung cancer or NSCLC.

In an embodiment, the present invention provides a method for detecting at least one gene modification, such as a mutation in a gene, such as a gene that codes for a protein associated with at least one of a tumor and a cancer. The method includes providing a detectable hybridization probe (sensor probe) which interacts with/binds to a gene not having a gene modification (wild type gene) and with a gene having a gene modification (mutation gene). The detectable hybridization probe (sensor probe) has at least one of a higher specificity, a higher binding affinity and a higher selectivity for the gene not having a gene modification (wild type gene) compared to the gene having a gene modification (mutation gene). At least one gene modification is detected with the detectable hybridization probe (sensor probe).

In an embodiment, the present invention provides a composition for use in the context of an asymmetric polymerase chain reaction (PCR) to detect at least one gene modification such as a mutation in a gene, such as a gene that codes for a protein associated with at least one of a tumor and a cancer. The composition includes a detectable wild type-specific hybridization probe (sensor probe). A first primer which binds at least substantially specifically to a single-stranded DNA of a mutation gene (mt-probe strand) with which the detectable wild-type-specific hybridization probe (sensor probe) can interact. A second primer which can interact at least substantially specifically with a single-stranded DNA of a mutation gene complementary to the probe strand (mt-complementary strand). A wild-type-specific blocking agent which inhibits a binding of the detectable wild-type-specific hybridization probe (sensor probe) to a wild type gene. At least one of a content and an amount of the first primer in the composition is greater than at least one of a content and an amount of the second primer in the composition.

In an embodiment, the present invention provides a method of using the aforementioned composition to detect at least one gene modification, such as a mutation in a gene, such as in a gene that codes for a protein associated with at least one of a tumor and a cancer. The method includes providing the aforementioned composition and using the aforementioned composition to detect at least one gene modification.

### BRIEF DESCRIPTION OF THE DRAWINGS

The present invention is described in greater detail below on the basis of embodiments and of the drawings in which:

FIG. 1 shows a schematic representation of the distribution or arrangement of the exon of the human EGF receptor gene in the protein encoded thereby and an overview of the mutations in the EGF receptor associated with drug resistance or drug sensitivity;

FIG. 2 shows the principle according to the present invention for detecting mutations using hybridization probes based on sensor probes which are specific to the wild type sequence of the corresponding mutation gene, on the one hand, and anchor probes on the other hand, and the formation of a FRET signal;

FIG. 3 shows the principle of the asymmetric PCR carried out according to the present invention;

FIG. 4 shows the principle of inhibition of the wild type or of the wild type DNA by the blocking agent used according to the present invention;

FIG. 5 shows specific melting curves for detecting an exon 19 deletion without adding a blocking agent;

FIG. 6 shows specific melting curves for detecting an exon 19 deletion using a blocking agent according to the present invention; and

FIG. 7 shows an overview of primers, probes and of the wild type-specific blocking agent usable according to the present invention and a schematic representation of the binding sites of the primer pairs used or tested and of the probes used.

### DETAILED DESCRIPTION

Configurations, embodiments, advantages and the like, which in the following, in order to avoid repetition, are only mentioned with respect to one aspect of the present invention, naturally also apply correspondingly to the other aspects of the present invention.

In an embodiment, the present invention provides a method for detecting at least one gene modification, in particular, a mutation, in a gene, for example, in a gene that codes for a protein associated with a tumor and/or cancer disease, in particular, wherein the gene that has the gene modification (mutation gene) is present together with further genes coding for the protein, but not having a gene modification (wild type genes), wherein the method according to the present invention is characterized in that for detecting the gene modification, a detectable hybridization probe (sensor probe) is used, which is capable of interacting with, especially binding to, the gene that does not have a gene modification (wild type gene) and the gene that has the gene modification (mutation gene), wherein the hybridization probe (sensor probe) has a higher specificity and/or binding affinity and/or selectivity for the gene that does not have a gene modification (wild type gene) than for the gene that has the gene modification (mutation gene).

The present invention is therefore based on the principle according to which, as it were, a wild type-specific hybridization probe is used as the hybridization probe or sensor probe, having a decreased specificity or affinity, especially binding affinity, for the gene that has the gene modification or mutation gene, in particular, caused by non-binding of the sensor probe at the position or site of the mutation. According to the present invention, a special sensor probe is thus used that is based on a nucleotide sequence that hybridizes more strongly to the corresponding single-stranded DNA of the wild type gene than to the corresponding single-stranded DNA of the mutation gene, and indeed, in particular, by binding of the sensor probe also in the site in the single-stranded DNA of the wild type gene corresponding to the mutation (i.e., also on the nucleotides or bases of the wild type sequence, which are located at the corresponding site or position as the mutation to be detected). Moreover, the sensor probe, as described in more detail below, according to the present invention is designed, in particular, so that, with respect to its length or number of nucleotides or bases, it comprises the region of the mutation and a nucleotide region in the gene adjoining the latter.

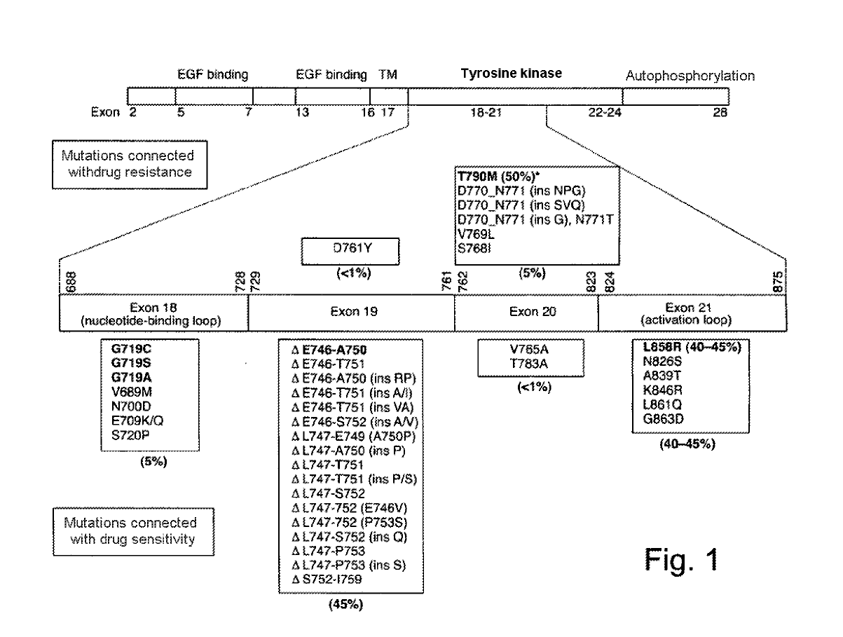

Owing to this principle underlying the present invention, it is possible to detect a large number of gene modifications occurring in a comparable position or site in the gene with one and the same sensor probe, which represents an enormous technical simplification, wherein the method according to the present invention leads, in particular, to a simple detection of mutations in the form of deletions or insertions and therefore of frameshift mutations at particular sites in the gene. Thus, for example, and non-limitatively, based on the method according to the present invention, it is possible, using a single type of sensor probe, to detect, for example, the deletions in exon 19 of the EGF receptor shown in FIG. 1. It is therefore possible according to the present invention, using a single type of sensor probe, to detect various deletions in exon 19 of the human EGF receptor, wherein the gene modification or deletions or insertions can have different sizes or slightly different localizations within the gene.

For example, it is possible according to the present invention to detect those deletions that lead to the omission of one, two, three or more amino acids in the amino acid sequence of the corresponding protein. Regarding the detectable mutations, especially deletions, it is therefore also possible to detect those changes that can result in the omission or the deletion of several amino acids. In this connection, it is possible, for example, and generally in the context of the present invention, to detect deletions that lead to the omission of three to fifteen or more nucleotides in the gene sequence coding for a protein. The omission of complete codons or individual nucleotides or bases (e.g., of one or two bases) can also be detected. These gene modifications, especially deletions, can moreover be detected with one and the same type of sensor, which are present at two or more successive positions with respect to the amino acid sequence of the protein in question. This includes, for example, deletions such as occur or begin at amino acid position 746 and/or 747 of the EGF receptor.

The sensor probe used according to the present invention can also be such that in the region before and/or after the actual mutation, the probe interacts with or hybridizes to the gene or the relevant single-stranded DNA, whereas in the region of the mutation there is no hybridization to the single-stranded DNA with the mutation. In contrast, the type of sensor probe used according to the present invention, as described in more detail hereunder, can hybridize at least substantially completely to the unmutated gene or the relevant single-stranded DNA without mutation. As a result, the respective complexes have different dissociation or melting temperatures, wherein the probe not hybridized completely to the single-stranded DNA with the mutation dissociates at lower temperatures than the sensor probe hybridized at least substantially completely to the single-stranded DNA of the wild type gene, which can be detected instrumentally from a change of a probe-specific detectable signal, as described in more detail hereunder.

The method according to the present invention is also suitable for detecting point mutations, for example, the T790M mutation in exon 20 of the EGF receptor described above, so that according to the present invention, as it were, through the respective tailoring of the sensor probe, a universally applicable method is provided for detecting mutations as such.

As described in more detail hereunder, the method according to the present invention is carried out, in particular, in connection with or by carrying out an asymmetric polymerase chain reaction, especially in combined use with a special blocking agent, which prevents or at least reduces the binding of the sensor probe used according to the present invention to the single-stranded DNA of the wild type, which leads to a marked improvement in the sensitivity of the method according to the present invention.

On this basis, another fundamental idea of the present invention is that owing to the specific design of the method according to the present invention, gene modifications or mutations in a sample or an ensemble of genes can be detected with very high sensitivity, so that even minuscule amounts of mutated DNA, especially in a mixture or ensemble with wild type DNA or DNA that does not have a mutation, can be analyzed. In the context of the present invention, it has therefore been possible to analyze even small amounts or contents of DNA which have the gene modification or mutation in a sample with high reliability and indeed, in particular, also to the extent that a large number of various kinds of mutations can be detected.

In this connection, it is possible according to the present invention to detect even tiny amounts of mutated DNA starting from a content of about 0.0025% in a mixture with other DNA or unmutated DNA or wild type DNA, relative to the DNA content.

Another fundamental idea of the present invention consists of increasing or improving the efficacy or sensitivity of the method according to the present invention in such a way that an asymmetric polymerase chain reaction is carried out in an appropriate way. Multiplication or amplification of the single-stranded DNA and especially of the mutation gene, which has the mutation and to which the sensor probe serving for the actual detection in the sense of an instrumental detection binds with lower affinity than to the single strand of the wild type gene, takes place as a priority or selectively. This can be achieved, for example, as described in more detail hereunder, by using different amounts of primers, which are also designated synonymously as (PCR) starter molecules, wherein, in the context of the present invention, in particular, the amount of the primer that binds to the so-called probe strand, in particular, with the mutation, is increased. As a result, according to the present invention, in particular, the amount of single strands of DNA with the mutation is increased compared to the other single strands of DNA so that, in the sample, the probe strand per se and, in particular, the DNA strand with the mutation is, as it were, overrepresented and therefore, owing to the statistically more frequent interaction with the sensor probe, an intensified sensor signal can be produced.

In the context of the present invention, it was surprisingly also possible to increase the sensitivity still further in that the asymmetric polymerase chain reaction can moreover be carried out in the presence of a special blocking agent or blocker, described in more detail hereunder, especially in the form of an oligo- or polynucleotide, especially in the form of LNA (locked nucleic acid) molecules or clamp molecules, which binds specifically, i.e., with increased affinity, to the single-stranded DNA without mutation or the wild type single-stranded DNA on the segment corresponding to the mutation region and prevents or reduces the binding of the sensor probe to this region of the unmutated DNA. As for the blocking agent or the LNA probe used, also designated synonymously as clamp probe or competitor, according to the present invention this is, for example, designed in such a way that the blocking agent, like the sensor probe used, binds to the wild type sequence, and indeed in a place where the sensor probe is also capable of binding. As a result, both the amplification of the corresponding single-stranded DNA on which the blocking agent binds, and the binding of the sensor probe to the corresponding wild type sequence, are minimized or suppressed.

Based on the combination of measures according to the present invention, it was surprisingly found to be possible to provide a method of detecting mutations or gene modifications which moreover leads to a marked intensification especially of the measurement signal due to the mutation to be investigated in the sense of a discrimination or intensification relative to the other signals in the sample and in which a large number of mutations can be detected with one and the same sensor probe. As a result, based on the method according to the present invention, even very small amounts of mutated DNA in a sample or starting material can be analyzed. Owing to the highly sensitive method, in the context of the present invention, it is possible to make use of samples that only have small amounts of DNA attributable to tumor cells. In the context of the present invention, even tiny proportions of mutated DNA can therefore be detected without having to carry out biopsies that are expensive and sometimes medically problematic. Basically, according to the present invention, the method according to the present invention can also be based on cell samples or cellular material, e.g., tumor cells as such.

Based on the method according to the present invention, with the informative detection or analysis of a large number of various kinds of mutations, which are of relevance with respect to a particular disease, it is furthermore also possible to provide, in a targeted way, information about the disease state or monitoring of the progression, in particular, with respect to the effects of drugs or the like, wherein, in addition, based on the concrete mutation analysis, relevant optimized tumor therapies, for example, using mutation specific inhibitors, can be carried out.

To summarize, in the context of the present invention, it is therefore possible for the first time, by using special hybridization probes in advantageous combination with an asymmetric polymerase chain reaction and using special blocking agents, to detect deletions of varying size and in a slightly different position, even with a small content of mutated DNA, in a sample to be analyzed.

The principle underlying the present invention is therefore a highly sensitive detection of various gene modifications or deletions by means of a single sensor probe. In contrast, up to now in the prior art it had only been possible to detect a single, specific point mutation by means of individual specific sensor probes. With respect to the EGF receptor, this is (purely as an illustration) possible, in particular, by specific binding of the sensor probe to the wild type sequence in the region of exon 19 of the EGF receptor. According to the present invention, the sensor probe used is therefore specially designed so that a nonspecific binding of the sensor probe takes place in the region of a potential deletion (mutation), wherein at the actual site of the mutation, advantageously there is at least substantially no interaction or binding. On this basis, according to the present invention, by far the commonest and best known mutations, especially in exon 19 of the EGF receptor, for example, beginning at amino acid position 746 or 747, can be detected.

The functioning of the sensor probe used according to the present invention can be illustrated hereunder purely as an example and without limiting the present invention, with a deletion in position 747 in exon 19 of the EGF receptor. If there is a deletion in this region, the sensor probe used according to the present invention is designed so that it binds in complementary fashion to the wild type sequence and terminates in position 747 with the last base or the last nucleotide. If there is now a deletion in one of the regions, the probe cannot bind, or can only bind inadequately, at position 747 or 746 and 747. In this way, any potential mutation or deletion in this region is detected with a single probe combination, especially as the sensor probe is also designed so that with respect to its length or the number of nucleotides or bases, it is larger than the deletion region to be detected. Therefore, the edge regions adjoining the mutation are also included, as it were, wherein in this respect, a region marginal or adjacent to the mutation is preferred, with which the sensor probe used also interacts in the case of the mutation gene, as this region corresponds to that of the wild type sequence. In the case of the aforementioned deletion at amino acid position 746 or 747, the blocking agent used is also, in particular, designed so that this also terminates with the last base of codon 747. In this way, the wild type is inhibited and the deletion can then be detected with high sensitivity with the sensor probe.

The present invention is not restricted to the detection of gene modifications in the EGF receptor, rather, in the context of the present invention, it is possible, in particular, to detect insertions, deletions and translocations as such in each case in different genes with high sensitivity. According to the present invention, consideration may moreover generally be given, in particular, to the detection of mutations at the sites known for the occurrence of chromosomal changes and that often recur. Clinically relevant examples of such mutations are, in addition to the EGF receptor, for example, insertions in the form of so-called Internal Tandem Duplications (ITD) in the receptor tyrosine kinase FLT3, which is often to be found in acute myeloid leukemia (about 30%). Translocations in the fusion gene or fusion protein EML4-ALK are other examples.

As mentioned above, in the context of the present invention, it is possible that the mutation gene or the mutation genes is/are present, as it were, in an ensemble or a sample together with a wild type gene or wild type genes, which is, for example, the case when both tumor cells and non-degenerated cells are present in the (initial) sample provided.

In the context of the present invention, it is also possible that the corresponding genes have or consist of mutation alleles and wild type alleles. For example, the mutation gene can, in the sense of a homozygotic manifestation, have two mutation alleles, i.e., both alleles of the gene are carriers of the corresponding gene modification or mutation. It is equally possible that the mutation gene is of heterozygotic form, wherein in this case an allele, namely, the mutation allele, is present with the gene modification or mutation and (corresponding to this) a wild type allele without mutation relative to the mutation gene. With respect to the wild type gene, which, in particular, is derived from healthy or nonmalignant cells, both alleles are, in particular, in the wild type form or as wild type alleles. To that extent, the terms “mutation gene” or “wild type gene” used hereinafter also refer, in particular, to the corresponding alleles, as defined above. The method according to the present invention is therefore also suitable for detecting mutations in the respective alleles of a gene.

In an embodiment of the present invention, the gene modification, in particular, a mutation, can be a frameshift mutation, especially a deletion and/or insertion. Equally, according to the present invention, it can be a point mutation. As mentioned above, the gene modification of the present invention can, for example, be a deletion and/or insertion. Equally, on the basis of the method according to the present invention, translocation mutations can also be detected.

It can be envisaged according to the present invention that the sensor probe is selected in such a way that the sensor probe is capable of interacting with, especially binding to, the single-stranded DNA (wt-probe strand) of the wild type gene and the corresponding single-stranded DNA (mt-probe strand) of the mutation gene having the gene modification, wherein the sensor probe has a higher specificity and/or binding affinity and/or selectivity for the single-stranded DNA (wt-probe strand) of the wild type gene than for the single-stranded DNA (mt-probe strand) of the mutation gene having the gene modification, especially at the position and/or site of the gene modification.

The sensor probe used according to the present invention is, in particular, such that it is capable of interacting with, especially binding to, the region of the wild type gene corresponding to the region of the gene modification, in particular, a mutation, of the mutation gene, in particular, wherein the interaction, especially binding, is weaker than to the corresponding region of the mutation gene. The sensor probe is, in particular, also designed so that with respect to its length or number of nucleotides, it comprises the region of the mutation at least partially and an adjoining edge region in the gene.

The sensor probe used according to the present invention can equally be selected so that the sensor probe has a lower specificity and/or binding affinity and/or selectivity for the single-stranded DNA (mt-probe strand) of the mutation gene, especially at the position and/or site of the gene modification, than to the corresponding single-stranded DNA (wt-probe strand) of the wild type gene. The term “corresponding single-stranded DNA of the wild type gene” relates, in particular, to the region of the single-stranded DNA without gene modification corresponding to the mutated single-stranded DNA.

The sensor probe used according to the present invention should, for example, be selected so that the sensor probe is capable of entering into specific binding with the single-stranded DNA (wt-probe strand) of the wild type gene, for example, being fully hybridized thereto.

Also regarding the sensor probe used according to the present invention, this should be selected so that the sensor probe is capable, at least substantially completely and/or substantially over its complete nucleotide sequence, of interacting with, in particular, binding to, the single-stranded DNA (wt-probe strand) of the wild type gene.

The length of the sensor probe or the number of nucleotides forming the sensor probe should accordingly be selected so that on the one hand it at least substantially comprises the number of nucleotides forming the gene modification and on the other hand has further nucleotides, which, as it were, form an edge region of the sensor probe or can bind to an edge region of the nucleotide sequence forming the gene modification.

The sensor probe should also be capable, at least substantially completely and/or at least substantially over its complete or whole nucleotide sequence, of interacting with, in particular, binding to, the segment of the single-stranded DNA (wt-probe strand) of the wild type gene that corresponds to the segment of the gene modification of the single-stranded DNA having the gene modification (wt-probe strand) of the mutation gene.

The sensor probe should also be selected so that the sensor probe has at least one substantially complementary nucleotide sequence to the single-stranded DNA (wt-probe strand) of the wild type gene. This provides at least substantially complete binding or hybridization of the sensor probe to the wild type DNA. The sensor probe should also be at least substantially complementary to the region of the wild type gene that corresponds to the mutation region, and an adjoining edge region.

Regarding the sensor probe used according to the present invention, the sensor probe of the present invention can, for example, also be capable of undergoing binding that is nonspecific and/or incomplete and/or in sections, to the single-stranded DNA (mt-probe strand) of the mutation gene, in particular, hybridizes to it only incompletely and/or only in sections.

In this connection, the sensor probe should be selected so that, at the position and/or site of the gene modification, the sensor probe is at least substantially not capable of undergoing binding to and/or interaction with the single-stranded DNA (mt-probe strand) of the mutation gene, in particular, at least substantially does not hybridize at a position and/or site of the gene modification.

The probe used according to the present invention should also be selected so that, at the position and/or site of the gene modification, the sensor probe is not capable of interacting with, in particular, is not capable of binding to the single-stranded DNA (mt-probe strand) of the mutation gene having the gene modification. In other words, the sensor probe is advantageously designed so that at the site or position of the mutation with the mutation gene, it is not capable of interacting with, in particular, binding to, the mutation gene. Accordingly the sensor probe at least substantially does not hybridize or at best only at the site or position of the gene modification.

It should be envisaged according to the present invention that the sensor probe is also selected in such a way that the sensor probe is only capable in sections of interacting with, in particular, binding to, the single-stranded DNA (mt-probe strand) of the mutation gene. In particular, the segment with which the sensor probe with the mt-probe strand is capable of interacting, is an edge segment or marginal region and/or an end segment or terminal region of the nucleotide sequence of the sensor probe. In other words, it can advantageously be envisaged according to the present invention that with respect to the sensor probe, as it were, the one end with a defined number of nucleotides or bases is capable of interacting with the mt-probe strand, whereas the other end with a defined number of nucleotides or bases does not interact with or hybridize to the mt-probe strand.

Based on this design of the sensor probe according to the present invention, it is accordingly, as it were, a “fluttering probe” with interaction with the mt-probe strand that is only marginal or in sections. According to the present invention, the sensor probe is thus advantageously a probe of a kind that is only capable of interacting with, in particular, binding to, the mutation gene with a portion and/or segment of its nucleotide sequence, wherein this refers in particular to an edge or end segment of the sensor probe.

The sensor probe should also be selected in such a way that the sensor probe is capable of interacting with, in particular, binding to, the segment of the single-stranded DNA (mt-probe strand) of the mutation gene having the gene modification adjoining the position and/or site of the gene modification.

It can also be envisaged according to the present invention that the sensor probe is selected in such a way that the sensor probe, relative to the single-stranded DNA (mt-probe strand) of the mutation gene having the gene modification, has at least one binding region and/or segment, for example, a single binding region and/or segment, and at least one non-binding region and/or segment, for example, a single non-binding region and/or segment. As the sensor probe, as mentioned above, hybridizes at least substantially completely to the wt-probe strand, relative to the wild type single-stranded DNA (wt-probe strand) the sensor probe accordingly has only a single, as it were continuous binding region, which is formed at least substantially by the complete nucleotide sequence of the sensor probe. In particular, the non-binding region of the sensor probe relates to the mutation region of the wt-probe strand, whereas the binding region relates to a region of the wt-probe strand which can, for example, directly adjoin the region of the gene modification, in particular, wherein this region of the wt-probe strand, to which the sensor probe can bind, has the wild type nucleotide sequence.

In general, the number of nucleotides of the non-binding region of the sensor probe can correspond at least substantially to the number of nucleotides forming the gene modification. For example, the non-binding region can comprise 3, 6, 9 or more nucleotides, wherein in this connection, integers between the aforementioned values are also possible, for example 1, 2, 4 or 5 nucleotides. According to the present invention, especially in the case of larger deletions, where two or more codons are deleted, it can also be envisaged that the non-binding region is smaller than the gene modification as such.

In other words, it can be envisaged that the number of nucleotides of the sensor probe which is not capable of interacting, especially not capable of binding to the single-stranded DNA (mt-probe strand) of the mutation gene having the gene modification, is in the range from 1 to 15, for example, 2 to 12, for example, 3 to 9, for example, 3 to 6. Since, as mentioned above, the sensor probe binds at least substantially completely to the wt-probe strand, the aforementioned values represent, as it were, the difference of binding nucleotides or bases of the sensor probe to the wt-probe strand and the binding nucleotides or bases of the sensor probe to the mt-probe strand.

In the same way, the number of nucleotides or bases of the binding region can be in the range from 1 to 30, for example, 3 to 21, for example, 6 to 12. Also in this connection, regarding the number of nucleotides forming the binding region, integers between the codon-forming triplet values are also possible, such as 4 or 5 nucleotides.

The sensor probe can in particular be selected in such a way that the sensor probe has at least 3, for example, at least 6, for example, at least 9 nucleotides and/or that the number of nucleotides of the sensor probe is in the range from 3 to 60, for example, 6 to 36, for example, 9 to 21. In this connection, integers between the codon-forming triplet values are also possible, such as 4 or 5 nucleotides. The size stated above relates to the nucleotide segment of the sensor probe.

The ratio of binding nucleotides to non-binding nucleotides of the sensor probe, relative to the single-stranded DNA (mt-probe strand) of the mutation gene having the gene modification, can be in the range from 10:1 to 1:10, especially 6:1 to 1:6, for example, 4:1 to 1:4. In other words, the ratio of binding region to non-binding region of the sensor probe used according to the present invention with respect to the respective number of nucleotides or bases can be based on the ratios stated above.

In the context of the method according to the present invention, it can be further envisaged that the sensor probe is selected in such a way that the sensor probe with at most 60%, for example, at most 50%, for example, at most 40%, of the nucleotides or bases forming the sensor probe is not capable of interacting with, in particular, not capable of binding to, the single-stranded DNA (mt-probe strand) of the mutation gene having the gene modification, relative to the total number of nucleotides or bases of the sensor probe. Accordingly it can be envisaged according to the present invention that the sensor probe with at least 40%, for example, at least 50%, for example, at least 60%, of the nucleotides or bases forming the sensor probe is capable of interacting with or binding to the single-stranded DNA (mt-probe strand) of the mutation gene having the gene modification. According to the present invention, the defined ratio of binding to non-binding nucleotides, relative to the mt-probe strand, leads to a differentiation of the sensor probe binding completely on the wt-probe strand of the wild type gene, especially with regard to the formation or tailoring of different dissociation or melting temperatures with respect to the hybridized sensor probes. In this way, differentiated information can be found about the presence of a mutation, which is namely manifested, in particular, by the presence of a lower melting temperature, due to the only partial hybridization of the sensor probe to the mt-probe strand, compared to the higher melting temperature of the sensor probe, which binds to the wt-probe strand.

In the context of the present invention, it can be envisaged in this connection, that the sensor probe is designed in such a way that a heat-induced detachment of the sensor probe from the single-stranded DNA of the mutation gene (mt-probe strand) takes place at lower temperatures than of the corresponding single-stranded DNA of the wild type gene (wt-probe strand). As mentioned above, this is provided or brought about, in particular, by the smaller number of nucleotides or bases binding on the mt-probe strand.

According to the present invention, the sensor probe can have or consist of the nucleotide sequence TAATTCCTTGATAGCGACGGG, relative to its nucleotide sequence. The aforementioned example is not limiting. Rather, a person skilled in the art is able at any time to select specific sensor probes with respect to their nucleotide sequence or their nucleotide segment, in such a way as to provide an increased specificity to the wild type gene in the sense of the present invention, and with respect to the mutations to be detected.

Regarding the binding of the sensor probe to the mt-probe strand, as an illustration this can be described in that the sensor probe binds in the region of the mt-probe strand directly adjoining the gene modification, whereas the mutation region is not involved in the binding, but the sensor probe overlaps the mutation region, as it were without undergoing binding.

Regarding the sensor probe (reporter probe) as such, this is a hybridization probe, which especially in the context of PCR and the related amplification steps, is capable of binding or hybridizing to the respective single-stranded DNA of the wild type gene or wild type allele and/or of the mutation gene or mutation allele in the region of the mutation to be analyzed or to be investigated, in particular, as described above.

An underlying principle of the present invention is also that the sensor probe used in the form of a hybridization probe possesses an increased selectivity or affinity for the single-stranded DNA of the wild type gene or wild type allele versus the corresponding gene segment or nucleotide region that has the mutation to be investigated. The selectivity or affinity of the sensor probe for the mutation region of the mutation gene or of the mutation allele or of the relevant single strand is therefore reduced. The selectivity or specificity with respect to the wild type region can be provided in a manner known per se by a person skilled in the art by special selection of the nucleotide sequence of the hybridization probe. In this connection, the nucleotide sequence or succession of bases should be selected so that the corresponding nucleotide sequence is at least substantially complementary to the corresponding nucleotides of the wild type. In this way, the increased sensitivity or affinity or strength of binding of the sensor probe with respect to the region of the wild type gene or allele corresponding to the mutation region will be provided insofar as the number of base pairs between sensor probe and wild type is greater than in relation to a thus far nonspecific interaction of the sensor probe with the corresponding gene segment of the mutation gene. Without wishing to be bound to this theory, the higher binding affinity, in particular, on the basis of the higher number of base pairs during interaction of the sensor probe with the corresponding gene segment or single-stranded DNA, leads to a “firmer” binding or interaction, which in comparison with the interaction of the sensor probe with the corresponding region of the mutation gene leads to a higher melting point, i.e., in particular, to a dissociation or de-hybridization of the sensor probe from the respective single-stranded DNA only at higher temperatures.

It can also be envisaged according to the present invention that the sensor probe is selected in such a way that in the case of interaction with and/or binding to the single-stranded DNA of the mutation gene (mt-probe strand) and/or to the corresponding single-stranded DNA of the wild type gene (wt-probe strand), the sensor probe is capable of emitting a detectable and/or measurable signal, in particular, a fluorescence signal. The signal can, in particular, also be a FRET signal, as is described in more detail below.

In the context of the present invention, it can also be envisaged that the sensor probe is designed in such a way that in the case of, in particular, heat-induced dissociation of the single-stranded DNA of the mutation gene (mt-probe strand) and/or of the corresponding single-stranded DNA of the wild type gene (wt-probe strand), the sensor probe cannot emit or else at least can only emit a reduced detectable and/or measurable signal, in particular, cannot emit or else at least can only emit a reduced fluorescence signal.

In the context of the present invention, the sensor probe can, for example, be a labeled nucleotide molecule, in particular, a labeled oligo- or polynucleotide, for example, provided with a detectable substance (tag), for example, with a substance that is detectable on interaction or binding of the sensor probe with the single-stranded DNA of the mutation gene, in particular, a dye, for example, a fluorescent dye.

In other words, the sensor probe should be arranged in such a way that it releases or emits a measurable signal, in particular, a fluorescence signal, at least substantially only on interaction with or binding or hybridization to the respective single-stranded DNA upon excitation, for example, upon excitation with electromagnetic radiation with a special wavelength or with a special wavelength range. Correspondingly, the non-bound sensor probe should not be able to emit a signal as such even upon excitation.

In particular, so-called FRET probes (fluorescence resonance energy transfer probes) can be used in the context of the present invention (especially in connection with the anchor probe presented below). It is possible, for example, to use various dyes, especially fluorescent dyes or fluorochromes, which are bound on the one hand to the sensor probe, and on the other hand to another molecule or another probe, such as the anchor probe. A signal can be emitted in this case as a function of the spacing of the corresponding fluorescent dyes and as a function of the binding to the respective single-stranded DNA. The relevant dyes or dye systems are familiar as such to a person skilled in the art.

According to the present invention, especially for purposes of formation of a FRET signal on interaction or binding of the sensor probe to the respective probe strand, it can be envisaged that, in addition to the sensor probe, at least one second hybridization probe different from it (anchor probe) is used, in particular, wherein the anchor probe should have a substance compatible with the detectable substance of the sensor probe for purposes of forming a FRET signal and/or that the sensor probe on the one hand and the anchor probe on the other hand, in particular, the detectable substance of the sensor probe on the one hand and the detectable substance of the anchor probe on the other hand, are capable of forming a FRET pair, especially for the case when both the sensor probe and the anchor probe are bound to the corresponding probe strand and/or when the anchor probe is selected so that the anchor probe is capable of binding to the same single-stranded DNA as the sensor probe, in particular, wherein the anchor probe is capable of binding at a distance of 1 to 5 bp (base pairs) from the sensor probe. In this connection, in the case of binding of the sensor probe and of the anchor probe to the corresponding probe strand, the detectable substances should be facing each other. The anchor probe should have a nucleotide or base sequence complementary to the wild type.

The detectable substances, in particular, substances forming the FRET pair, can be coupled to the corresponding probes in a manner known per se by a person skilled in the art, for example, with a covalent bond.

In an embodiment of the present invention, in addition to the sensor probe, at least one second hybridization probe different from it (anchor probe) can be used.

In this connection, in the context of the present invention, the anchor probe can, for example, be a nucleotide molecule, for example, provided or labeled with at least one detectable substance (tag) that is, in particular, complementary to or compatible with the sensor probe, especially a dye, for example, a fluorescent dye. The anchor probe should be an oligo- or polynucleotide or should have such a molecule or such a segment. The nucleotide segment of the anchor probe should have a size from 3 to 50 bp, for example, 5 to 45 bp, for example, 10 to 40 bp. The nucleotide sequence of the anchor probe should be selected in such a way that the anchor probe is capable of binding to the same single-stranded DNA as the sensor probe, wherein the binding of the anchor probe should take place in a region adjacent to the gene modification, in particular, mutation, to be analyzed and therefore not in the immediate mutation region or the corresponding site of the wild type single-stranded DNA. According to the present invention, the binding of the anchor probe to the respective single-stranded DNA, in particular, to the probe strand, should take place in such a way that substances to be detected, especially dyes, of the anchor probe on the one hand and of the sensor probe on the other hand are capable of interacting with one another to form a detectable signal, for example, in the form of emitted light, especially in the manner of a FRET pair.

It can be envisaged according to the present invention that the anchor probe is capable of binding to the same single-stranded DNA as the sensor probe, in particular, wherein the anchor probe should be capable of binding to the sensor probe at a distance of 1 to 5 bp. Interaction of the detectable substances, especially of the fluorescent dyes, becomes possible in this way. The aforementioned distance of 1 to 5 bp corresponds to a spatial gap of about 1 to 10 nm. As mentioned above, in the context of the present invention, the anchor probe can be arranged so that it is capable of binding both to the single-stranded DNA with the mutation (probe strand) and to the corresponding wild type single-stranded DNA. This is also achieved in that the respective binding regions, relative both to the probe strand and to the wild type single-stranded DNA in the region of the binding site of the anchor probe, have an at least substantially identical nucleotide sequence or succession of bases. Against this background, the anchor probe can bind with comparable specificity to the probe strand on the one hand and the wild type single-stranded DNA on the other hand, whereas the sensor probe binds with increased affinity to the probe strand compared to the wild type single-stranded DNA. In the context of the present invention, it is advantageous if the sensor probe on the one hand and the anchor probe on the other hand, especially the detectable substance or tag of the sensor probe on the one hand and the detectable substance or tag of the anchor probe on the other hand, are capable of forming a FRET pair during binding of the probes.

In this connection, there is a possibility of using FRET for quantifying the underlying gene modification or mutation in the use of so-called LightCycler® probes, which are special hydrolysis probes, wherein various oligonucleotides, each labeled with a donor or acceptor (sensor probe and anchor probe), which bind next to one another to the target sequence (wild type or mutation region relative to the sensor probe on the one hand and the respective adjacent region relative to the anchor probe) of the gene segment to be investigated or the corresponding single-stranded DNA and thus bring the donor and the acceptor sufficiently close together for FRET. Probe pairs of this kind can therefore be used for quantifying the underlying mutation in the context of the method according to the present invention and therefore as it were for quantifying PCR products. Moreover, in the context of the present invention it can be envisaged that for example the sensor probe is provided or labeled with a detectable substance, especially fluorescent dye, in the form of a donor and the anchor probe correspondingly with a detectable substance, especially fluorescent dye, in the form of an acceptor. Provision or labeling the other way round, i.e. providing the sensor probe with an acceptor and the anchor probe with a donor, is also possible in the context of the method according to the present invention.

Without wishing to be tied to a theory, during binding of the sensor probe on the one hand and of the anchor probe on the other hand to the respective DNA strand, donor and acceptor are brought spatially close together to produce FRET with corresponding excitation, which leads to a detectable signal. On dissociation of the sensor probe or of the anchor probe with concomitant spatial separation, even upon excitation, FRET does not occur, so that in this case no FRET signal is detectable. The underlying principle of FRET or of signal formation with the specific use of donor and acceptor molecules can therefore be used as a measure for the binding of sensor or anchor probes to a respective DNA segment. The fluorescence moreover increases in proportion to the concentration of complementary DNA, i.e., the more sensor probes or anchor probes are bound to the DNA of the PCR preparation, the stronger is the fluorescence signal.

In an embodiment of the present invention, with respect to the probe pair to be used, the sensor probe can be a LightCycler® probe with the nucleotide sequence TAATTCCTTGATAGCGACGGG described above, to which a fluorescent dye is coupled as part of a FRET pair (provisional Sequence Listing II, SEQ ID NO. 4). The anchor probe can also be a LightCycler® probe, which has the nucleotide sequence ATTTTAACTTTCTCACCTTCTGGGATCCAG and is also provided with a fluorescent dye (provisional Sequence Listing II, SEQ ID NO. 5).

On account of the bound fluorescent dye, this sensor probe is able, in particular, to emit a detectable and/or measurable signal with an anchor probe in the case of hybridization to the single-stranded DNA of the wild type gene or wild type allele (wt-probe strand) and/or in the case of, in particular, nonspecific hybridization to the corresponding single-stranded DNA of the mutation gene (mt-probe strand).

The signal to be detected, which occurs on interaction with the corresponding single-stranded DNA, can be measured, for example, at the end of an annealing phase of a PCR cycle. In addition, the fluorescence, as described below, can be determined or detected in the context of a melting curve analysis.

In the case of detection or analysis of the aforementioned deletions in exon 19 of the EGF receptor, especially at positions 746 or 747 of the amino acid sequence of the EGF receptor, the anchor probe can, non-limitatively, have or consist of the nucleotide or base sequence ATTTTAACTTTCCACCTTCTGGGATCCAG.

In the case of detection or analysis of mutation T790M, the anchor probe can, in particular, have or consist of the nucleotide or base sequence CACGGTGGAGGTGAGGCAGATGC. In the case of analysis or detection of mutation L858R, the nucleotide or base sequence of the anchor probe comprises or consists of the nucleotide or base sequence GCATGGTATTCTTTCTCTTCCGCACCCAGC.

A person skilled in the art is capable at any time of correspondingly selecting or tailoring the anchor probe specially to be formed or to be used for detecting the respective mutation.

In the present invention, the tumor or cancer disease can moreover be a lung carcinoma, in particular, a non-small cell lung cancer (NSCLC) and/or a small cell lung cancer (SCLC), for example, a non-small cell lung cancer (NSCLC).

In other words, the present invention focuses on detecting mutations that are associated with the aforementioned cancer diseases, in particular, associated with a non-small cell lung cancer, or cause these diseases, for example, through abnormal functioning of the resultant gene product of the mutation gene.

The present invention is not, however, restricted to the aforementioned diseases. Rather, the method according to the present invention can, as it were, be used universally for analysis or detection of, in particular, known mutations or gene modifications that are associated with diseases of the organism as such. This can be achieved, for example, by specific adaptation of the hybridization probes or of the sensor probes, of the primers and/or of the blocking agent, so that the method according to the present invention can, as it were, be tailored to a large number of specific diseases.

The protein in question, the modification of which can be determined by detecting or analyzing the mutation of the gene coding for the protein, according to an embodiment of the present invention is a protein that is, in particular, human and/or regulates and/or induces cell growth and/or cell proliferation. The protein can in addition be an, in particular, transmembrane receptor for growth factors, in particular, with intrinsic tyrosine kinase activity. The protein in question, which is associated with a tumor or cancer disease, is for example, the epidermal growth factor receptor (EGF receptor), in particular, as described above. In particular, in the context of the present invention, it is the human EGF receptor.

The present invention also equally comprises structure-identical or structure-similar proteins with action identical or similar to the aforementioned EGF receptor. The term “EGF receptor”, as used in the context of the present invention, in particular, also comprises modifications with identical or similar function as well as mutation forms of the EGF receptor itself, not directly associated with the cancer or tumor disease. In particular, the term “EGF receptor” also, in particular, comprises isoforms and/or precursors with at least substantially similar or identical function. In an embodiment of the present invention, the protein in question can, for example, be the EGF receptor according to the locus or according to reference number NP_{—}005219.2 in Sequence Listing I and/or the amino acid sequence listed in Sequence Listing II under SEQ ID NO. 1.

In general, the present invention also comprises such forms of the EGF receptor or proteins in general that have agreement in the amino acid sequence of at least 90%, for example, at least 95%, for example, at least 98%, for example, at least 99%, relative to the amino acid sequence of the EGF receptor according to reference number NP_{—}005219.2 in Sequence Listing I and/or according to SEQ ID NO. 1 in Sequence Listing II.

Moreover, Sequence Listing I is synonymous or identical in content to Sequence Listing II with respect to the amino acid sequence listed as NP_{—}005219.2 or SEQ ID NO. 1. The essential difference between the sequence listings is that Sequence Listing I is based on a scientifically standardized statement or representation, whereas Sequence Listing II was prepared on the basis of a statement or representation standardized in patent law using the PatentIn software, version 3.3. Thus, with respect to the amino acid sequence, this is a purely formal difference in representation, but not a difference in content.

The gene modification, in particular, mutation, to be investigated or to be analyzed in the context of the method according to the present invention can therefore lead to and/or can be associated with an increased and/or excessive activity of the protein encoded by the corresponding gene. The gene modification or mutation can therefore lead to or be associated with increased or excessive cell growth and/or increased or excessive cell proliferation. The gene modification to be investigated can lead to activity of the protein encoded by the corresponding gene, in particular, of the EGF receptor, that is pathological or deviates from the physiological norm. In other words, the gene modification, in particular, mutation, brings about a change in the corresponding gene product, which is associated with the tumor or cancer disease. The disease that is associated with the gene modification is, in particular, as mentioned above, a bronchial carcinoma, in particular, NSCLC.

The gene modification can, in particular, be a gene modification in exon 18, exon 19, exon 20 or exon 21, for example, in exon 19, of the epidermal growth factor receptor (EGF receptor).

In particular, the gene modification can be a gene modification, especially deletion, in exon 19 and/or in the region of the amino acid position 746 and/or 747 of the epidermal growth factor receptor (EGF receptor). As mentioned above, the deletion can be the omission of complete codons based on three nucleotides or bases. Equally, however, the omission of individual bases, for example, of one base or of two bases, is also possible.

The gene modification in question can also be a deletion, in particular, in exon 19 of the Epidermal Growth Factor receptor (EGF receptor), wherein the deletion can be selected from the group of deletions ΔE746-A750, ΔE746-T751, ΔE746-A750 (ins RP), ΔE746-T751 (ins A/I, ΔE746-T751 (ins VA), ΔE746-S752 (ins A/V), ΔL747-E749 (A750P), ΔL747-A750 (ins P), ΔL747-T751, ΔL747-T751 (ins P/S), ΔL747-S752, ΔL747-S752 (E746V), ΔL747-S752 (P746V), ΔL747-S752 (ins Q), ΔL747-P753, ΔL747-P753 (ins S) and ΔS752-I759.

According to the present invention, gene modifications that lead to and/or are associated with a deletion, in particular, in exon 19 of the EGF receptor, can therefore be detected. Equally, it is possible to detect gene modifications that lead to or are associated with a substitution of at least one amino acid in exon 20 and/or exon 21 of the EGF receptor. In this connection, it is possible, for example, to detect gene modifications that lead to or are associated with substitution of serine in position 768 of the EGF receptor, in particular, with isoleucine (S768I). Similarly, gene modifications can also be detected that lead to or are associated with substitution of threonine in position 790 of the EGF receptor, in particular, with methionine (T790M). In addition, gene modifications can also be detected that lead to or are associated with substitution of leucine in position 858 of the EGF receptor, in particular, with arginine (L858R).

As mentioned above, the present invention is not limited to the detection of mutations in the aforementioned EGF receptor. Equally, gene modifications, in particular, in the form of deletions or insertions, can be detected for a large number of other proteins, which, in particular, are associated with cancer or tumor diseases.

In this connection, the tumor and/or cancer disease can be associated with a gene modification, in particular, translocation, in the gene coding for the fusion protein EML4-ALK. The gene modification can therefore represent a gene modification, in particular, translocation, in the gene coding for the fusion protein EML4-ALK.

In the context of the present invention, the tumor or cancer disease can also be a leukemia, in particular, an acute myeloid leukemia (AML). In this connection, the tumor or cancer disease can be associated with a gene modification, in particular, insertion, in the gene coding for receptor tyrosine kinase FLT3. Accordingly, the gene modification can represent a gene modification, in particular, insertion, in the gene coding for receptor tyrosine kinase FLT3. In particular, it is the aforementioned internal tandem duplication (ITD).

In addition to the aforementioned deletion, in particular, in the case of non-small cell bronchial carcinoma, the present invention also focuses on clinically-related examples of other gene modifications that are associated with cancer diseases. An example of relevant translocations is the aforementioned fusion gene or fusion protein EML4-ALK, whose occurrence is also associated with noncellular bronchial carcinoma. As well as carcinomas of the lung, other malignant diseases are also associated with the occurrence of gene modifications, which can be detected in the context of the method according to the present invention, in particular, those of a hematological nature. An example is the aforementioned acute myeloid leukemia (AML), which is a malignant disease of the hematopoietic system (i.e., of hematopoiesis) in particular, myelopoiesis. Myelopoiesis denotes the part of hematopoiesis that comprises the differentiation of pluripotent cells into granulocytes, erythrocytes, thrombocytes, macrophages and mast cells. Acute myeloid leukemia (AML) leads to a massive multiplication of immature precursors (i.e., of not fully differentiated forms of the aforementioned cell types) in the bone marrow and often also in the blood. Similarly to the link between bronchial carcinomas and gene modifications of the gene coding for the EGF receptor, also in the case of AML an association of the occurrence of the disease with the gene modification of the FLT3 gene, which codes for an Fns-like tyrosine kinase from the family of class III tyrosine kinase receptor proteins, can be detected. About 30% to 40% of adult AML patients thus display modifications of the FLT3 gene. The gene modifications relevant to AML, which can be detected in the context of the method according to the present invention, can be both internal tandem duplication (ITD) of the gene segment coding for the juxtamembrane domain and ITDs that are located in regions outside of the juxtamembrane domain.

It can further be envisaged that the method according to the present invention is carried out by means of an asymmetric polymerase chain reaction (PCR) using at least one sensor probe and optionally at least one anchor probe and optionally at least one wild type-specific blocking agent inhibiting the binding of the sensor probe to the wild type gene. Moreover, in the context of the present invention, the procedure followed can, in particular, be that a selective multiplication or amplification of those DNA single strands also of the mutation gene (mt-probe strands) and of the wild type gene (wt-probe strands), with which the sensor probe is capable of interacting, in particular, binding thereto, takes place.

In the context of the present invention, it can in addition be envisaged that the asymmetric PCR is carried out in the presence of primers, in particular, in the form of oligonucleotides.

In this connection, the primers can be selected so that, in particular, in the context of PCR an amplification of the gene segment of the mutation gene or mutation allele having the gene modification takes place and/or so that an amplification of the gene segment of the wild type gene or of the wild type allele corresponding to the gene segment of the mutation gene having the gene modification takes place. The term “the gene segment of the wild type gene corresponding to the gene segment of the mutation gene having the gene modification” is to be understood, in particular, as meaning the segment of the wild type gene or of the wild type allele, in which there is no mutation as such, corresponding to the segment with the mutation.

Through selection of the primers, targeted amplification or multiplication of the gene segment relevant to the investigation or genetic analysis, or of the gene segment of the wild type gene corresponding to this, and therefore discrimination against the other genes or the other DNA in the sample, is possible. In particular, the DNA to be amplified is the gene of the EGF receptor described above or, for example, a gene segment of the gene coding for the EGF protein, and indeed, in particular, the gene segment in which the mutation can be present. The concrete selection of the relevant primer to be used does not pose any problem for a person skilled in the art, and a person skilled in the art is able at any time to select and use the corresponding primer.

The primers used in the context of the present invention can be selected so that there is amplification of the gene segment of the mutation gene having the deletion, in particular, in exon 19 of the EGF receptor, in particular, in position 746 and/or 747. In the context of the present invention, it can also be envisaged that the primers are selected so that there is amplification of the gene segment of the mutation gene having the substitution of serine in position 768 of the EGF receptor, in particular, with isoleucine (S768I). In an embodiment of the present invention, the primers can be selected so that there is amplification of the gene segment of the mutation gene having the substitution of threonine in position 790 of the EGF receptor, in particular, with methionine (T790M), and/or there is amplification of the gene segment of the mutation gene having the substitution of leucine in position 858 of the EGF receptor, in particular, with arginine (L858R).

In this connection, it can also optionally be possible that there can simultaneously be amplification of the gene segment of the wild type gene corresponding to the gene segment of the mutation gene having the gene modification, in particular, owing to the properties of the primers as such.

Regarding the gene segments to be amplified, the relevant size or the number of base pairs should be selected so that the sensor probe and the anchor probe optionally equally binding to the gene segment are capable of binding to the corresponding single-stranded DNA of the respective gene segments.

In the context of the present invention, it can be envisaged that a first primer and a second primer, different from the first primer, are used. In this connection, the first primer should bind at least substantially specifically to the single-stranded DNA of the mutation gene (wt-probe strand), to which the sensor probe is capable of binding or hybridizing, or with which the sensor probe is capable of interacting. The first primer can, owing to the nature of the primers, also bind to the corresponding single-stranded DNA of the wild type gene. The first primer is therefore as it were a sense-primer. Regarding the second primer, this should bind at least substantially specifically to the single-stranded DNA of the mutation gene complementary to the probe strand (mt-complementary strand) or be capable of binding to it or interacting with it. The second primer is as it were an antisense-primer. The second primer can, moreover, bind to the relevant corresponding single-stranded DNA of the wild type gene.

In the case of the special detection or analysis of the listed deletions in exon 19 of the EGF receptor, the first and second primer should be selected so that specifically the gene segment of the mutation gene or of the corresponding wild type gene having the mutation is amplified in the context of the PCR; this applies correspondingly to the analysis or detection of the mutation mutation T790M or L858R.

In the case of analysis or detection of deletions in the region of amino acid position 746 or 747 in exon 19 of the EGF receptor, the first primer can, non-limitatively, in particular, have the base sequence or nucleotide sequence GGGCCTGAGGTTCAGAGC (cf. Sequence Listing II, SEQ ID NO. 3) or CACACAGCAAAGCAGAAACTCA or consist of this nucleotide sequence. In the case of the second primer, this can, non-limitatively, have or consist of the nucleotide sequence GTCTTCCTTCTCTCTCTGTCATAGGG (cf. Sequence Listing II, SEQ ID NO. 2) or ATCTCACAATTGCCAGTTAACGTCT.

In the case of analysis or detection of mutation T790M, the first primer can, non-limitatively, in particular, have the succession of bases or the nucleotide sequence GACTCCGACTCCTCCTTTATCCAATG or consist of this nucleotide sequence. In the case of the second primer, this can, non-limitatively, have or consist of the nucleotide sequence CACACACCAGTTGAGCAGGTA.

In the case of analysis or detection of mutation L858R, the first primer can have or consist of the nucleotide sequence GCTCAGAGCCTGGCATGAA; the second primer can have or consist of the nucleotide sequence CATCCTCCCCTGCATGTGT.

The respective primers, in particular, also comprise those primers that have a comparable specificity or selectivity with respect to the respective gene segments. This is, however, known per se by a person skilled in the art, and a person skilled in the art is able at any time, against the background of amplification of the corresponding gene segments, to select the specific primers in each case. The primers should be selected so that these even bind outside of the region of the mutation of the mutation gene to be analyzed or of the corresponding segments of the wild type gene or the relevant DNA single strands.

In an embodiment of the present invention, the asymmetric PCR can, for example, be carried out in such a way that the single-stranded DNA, to which the sensor probe (probe strand) is capable of binding, is amplified more strongly or more frequently than the single-stranded DNA that is complementary to it (complementary strand), so that after amplification there are more copies of the probe strand compared to the complementary strand in the PCR mixture. In the context of the present invention, according to an embodiment of the present invention, this can, for example, occur because the first primer and the second primer are selected so that the amount and/or the concentration of the first primer, in particular, relative to the PCR mixture, is greater than the amount and/or concentration of the second primer, in particular, so that there is increased and/or intensified amplification of the probe strand versus the complementary strand. In this way there is intensified amplification of the probe strand versus the complementary single-stranded DNA, with the result that the single-stranded DNA, to which the sensor probe is capable of binding, is present in a higher copy number, so that owing to the larger number of events relative to the binding between sensor probe on the one hand and corresponding single-stranded DNA on the other hand, there can be intensification or amplification of the mutation-specific sensor probe signal.

In this connection, in the context of the present invention, the quantitative ratio of the first primer to the second primer (first primer: second primer), in particular, in the PCR mixture, can, for example, be in the range from 1000:1 to 1.05:1, for example, 100:1 to 1.5:1, for example, 10:1 to 2:1.

In an embodiment of the present invention, after completion of amplification, the number or concentration of the probe strand versus the complementary strand can, for example, be increased by a factor of at least 1.1, for example, 1.5, for example, 2, for example, 10, for example, 100.

As mentioned above, through the special ratio of the primers, for example, the first primer, in the context of the PCR-based amplification, primarily the single-stranded DNA of the mutation gene or of the mutation allele is amplified or multiplied. In this way the mutation-specific signal is additionally intensified.

The applicant also surprisingly found that the limit of detection or sensitivity of the method according to the present invention for detection or analysis of particular mutations can be further increased significantly by carrying out the asymmetric PCR and the associated use of wild type-specific hybridization probes in a special combination with the use of at least one blocking agent. Entirely surprisingly, this can give a further increase in sensitivity, wherein the totality of the measures envisaged according to the present invention (use of specific sensor probes, asymmetric PCR and use of special blocking agents) goes beyond the effect of the individual measures, so that the measures taken according to the present invention surprisingly act synergistically with respect to the improvement of the sensitivity of the method according to the present invention.

In an embodiment of the present invention, the blocking agent can, for example, be selected so that the blocking agent has a higher specificity or binding affinity or selectivity with respect to the single-stranded DNA of the wild type gene or wild type allele (in particular, with respect to the region of the single-stranded DNA of the wild type gene or wild type allele, which corresponds to the gene segment of the mutation gene or mutation allele having the gene modification (wild type DNA strand)) versus the corresponding mutation gene or the relevant single-stranded DNA with the gene modification or mutation (mutated DNA strand). In other words the blocking agent should be selected so that it binds with higher specificity or selectivity to the wild type DNA strand and, in particular, to the position or site corresponding to the mutated single-stranded DNA, so as in this way to reduce or prevent the binding of the sensor probe to the wild type DNA strand. Blocking agent on the one hand and sensor probe on the other hand therefore behave competitively with respect to the binding sites, wherein the binding of the sensor probe to the wild type DNA strand is reduced or prevented. In this way there can be further discrimination or intensification of the signal to be detected of the sensor probe relative to the mutation to be detected, as fewer sensor probes bind to the wild type DNA strand if the blocking agent is present in the PCR mixture. With respect to the mutated DNA strand, the sensor probe has a higher affinity than the blocking agent, so that the binding of the sensor probe to the mutated DNA strand is at least not substantially influenced or prevented by the blocking agent.

In this connection, the binding and/or the complex of the blocking agent with the single-stranded DNA of the wild type gene should have a higher stability, in particular, a higher melting point, than the binding or the complex of the sensor probe with the single strand of the mutation gene. This can be provided in the context of the present invention because the nucleotide sequence of the blocking agent is at least substantially complementary to the unmutated region of the wild type single-stranded DNA corresponding to the mutation region of the mutated single-stranded DNA.

In this connection, in an embodiment of the present invention, the blocking agent can, for example, be selected so that it has or consists of a nucleotide molecule, in particular, an oligo- or polynucleotide. Moreover, the blocking agent should have a size from 3 to 30 bp, for example, 5 to 25 bp, for example, 10 to 20 bp.

As mentioned above, the blocking agent should be selected according to the present invention so that the blocking agent is at least substantially complementary to the single-stranded DNA of the wild type gene or wild type allele, in particular, to the region of the single-stranded DNA of the wild type gene or wild type allele that corresponds to the gene segment of the mutation gene or mutation allele having the gene modification. The region of the wild type single-stranded DNA, which corresponds to the gene segment of the mutation gene having the gene modification or to the segment of the single-stranded DNA having the mutation, therefore represents as it were the segment of the wild type DNA strand of the wild type gene analogous to the mutation region, on which the blocking agent or the blocker is capable of binding specifically.

As a result of using the specific blocking agent, the binding of the sensor probe to the wild type strand is therefore inhibited or reduced, so that only or primarily the mutated DNA strand is multiplied in the context of the PCR and the probes used can bind without competition to the mutated DNA. The blocking agent therefore prevents or reduces on the one hand the amplification of the wild type DNA strand and on the other hand the binding of the sensor probe to the wild type DNA strand, in particular, in the sense of competitive binding, which further improves the measurement result.

For this purpose, the blocking agent should therefore in general be such that it has very high affinity for the wild type single strand and only melts at very high temperatures. In this connection, the blocking agent should be selected so that the blocking agent has bridged nucleic acids, in which the sugar moiety, in particular, the ribose moiety, is chemically modified, in particular, wherein the ribose moiety has an oxygen/methylene bridge, for example, on the C₂- and C₄-atom of the ribose moiety.

In this connection, the blocking agent can be selected so that the blocking agent is a nucleic acid analog in the form of a locked nucleic acid (LNA). These locked nucleic acids are in particular molecules that are structurally less flexible and therefore stiff against torsion, which bind or hybridize specifically to the DNA strand, giving rise to a higher melting point.

In the case of detection or analysis of the aforementioned deletions in exon 19 of the EGF receptor, in particular, at positions 746 or 747 of the amino acid sequence of the EGF receptor, the blocking agent, in particular, in the form of an LNA, can have or consist of the nucleotide sequence TAATTCCTTGATA (cf. Sequence Listing II, SEQ ID NO. 6).

In this connection, with reference to detection or analysis of mutation T790M, the blocking agent, in particular, in the form of an LNA, should have or consist of the nucleotide sequence TGAGCTGCGTGATG; with respect to detection or analysis of mutation L858R, the blocking agent, in particular, in the form of an LNA, should have the nucleotide sequence GCCAGCCCAAAATCT or consist of this nucleotide sequence.

In the context of the present invention it is equally possible that the blocking agent is another nucleic acid analog, in particular, wherein the blocking agent has a peptide and/or peptide-based backbone and/or, in particular, wherein the blocking agent is a peptide nucleic acid (PNA). This is a DNA analog in which the sugar-phosphate backbone is replaced with a pseudopeptide. Peptide nucleic acids of this kind also lead to a higher affinity of binding to the complementary DNA sequence, which leads to the formation of firmer or more stable bonds, together with an increase in melting point.

For detecting or deleting the gene modification, in particular, following the polymerase chain reaction, a melting curve can be recorded or a melting curve analysis can be carried out. In this connection, the cleavage or dehybridization, in particular, of the sensor probe, from the respective single-stranded DNA of the mutation gene or of the wild type gene can be detected, in particular, wherein the detection can take place photometrically, in particular, by measuring the fluorescence. A conclusion can be drawn regarding the presence of a mutation from the melting points and/or the melting point range of the melting curve. Detachment of the sensor probe leads to a decrease or reduction of the detectable signal, for example, in the case of a FRET pair using an anchor probe through spacing of the detectable substances of the sensor and anchor probe forming the FRET pair. As mentioned above, in the case of the mutation, this occurs at lower temperatures compared to the wild type.

In an embodiment of the present invention, the method can be carried out so that for detecting the gene modification, in particular, the mutation, in particular, following the polymerase chain reaction, a melting curve is recorded or a melting curve analysis is carried out. In this connection, the reaction mixtures can be heated slowly, for example, up to 95° C. At the point or at the temperature at which 50% of the sensor probes have detached from the resultant PCR products, there is a marked decrease in fluorescence. If the sensor probe has bound specifically to the wild type single-stranded DNA, the dehybridization of the sensor probe takes place at a higher temperature compared to binding to the corresponding mutation region or the corresponding mutation single-stranded DNA. The corresponding analysis of the melting curves therefore allows conclusions to be drawn concerning the presence of a specific mutation.

In this connection, the cleavage or dehybridization, in particular, of the sensor probe, from the respective single-stranded DNA of the mutation gene or of the wild type gene can be detected, in particular, wherein the detection takes place photometrically, in particular, by measuring the fluorescence. As shown above, the hybridization or the cleavage of the sensor probe from the respective single-stranded DNA leads to a decrease of fluorescence, wherein, owing to the higher affinity with respect to the wild type single-stranded DNA, the associated melting point is higher than for the single-stranded DNA with the corresponding mutation. By using the sensor probes, it can accordingly be established whether mutated gene forms are present in a sample. For better analysis of the melting curves, these can be presented in the form of the first mathematical derivative, in particular, the maxima of the relevant first mathematical derivative represent the respective melting points.

In the context of the method according to the present invention, the procedure followed can therefore be that based on the melting points and/or melting point ranges of the melting curve, a conclusion can be drawn about the presence of a gene modification, in particular, a mutation. In other words, on the basis of the analysis or assignment (i.e., assignment via a reference, as described in more detail below) of the melting points or melting ranges, it can be established whether, and if in the affirmative, what concrete mutation is present in the sample.

In this connection, for example, the procedure followed can be that in the context of the melting curve analysis, parallel comparative or reference assays are conducted simultaneously or analyzed or alternatively these comparative or reference assays have been measured beforehand and/or independently (i.e., in other words, the method is standardized via a reference). At least one reference assay based on a wild type DNA or a wild type gene or allele can, for example, be conducted simultaneously in an, in particular, parallel assay or measured or standardized beforehand. The reference assay can be conducted or set up without a blocking agent, i.e., a further reference assay of the wild type gene can be conducted simultaneously without blocking agent. It can also be envisaged that at least one reference assay based on a gene modification or mutation to be analyzed and/or a defined or previously known gene modification or mutation is conducted simultaneously in an, in particular, parallel assay or is measured or standardized beforehand; these can, in particular, be reference assays that contain mutation genes or mutation alleles based on the aforementioned deletions in exon 19 of the EGF receptor.

By comparing the melting curves obtained on the basis of the samples to be analyzed on the one hand, and on the basis of the reference assays or measurements on the other hand, it is then possible to obtain information about the presence of a special mutation in the sample (i.e., testing for the presence of a gene modification or mutation and, if in the affirmative, the nature of the gene modification or mutation). It can then generally be assumed that a gene modification or mutation is present if, relative to the reference assay with the wild type, different melting points or melting ranges, in particular, occurring at lower temperatures, are found for the sample to be investigated. The presence of a gene modification or mutation can also be found by comparing the melting curve with the sample of the respective reference assay with the relevant mutation gene, wherein the presence of at least substantially equal melting points or ranges can be taken as a sign for the presence of the same gene modifications or mutations.

In an embodiment of the present invention, it is also possible to conduct the analysis for the presence of a mutation without a reference assay, in particular, if the melting points or ranges are already known or if standardization has been carried out beforehand (in particular, as described above).

As mentioned above, it can be envisaged according to the present invention that the mutation gene or the mutation genes on the one hand and the wild type gene or the wild type genes on the other hand are present in a sample and/or an ensemble, in particular, wherein the sample provided and/or the ensemble originate from a patient and/or, in particular, wherein the sample and/or the ensemble originate from a body material, in particular, a body fluid, for example, blood and/or tissue fluid and/or lymph and/or urine with, in particular, cellular constituents, or can be obtained therefrom. In addition, prepared cellular material or tumor material can also be used, for example, based on a biopsy.

The sample on which the method according to the present invention is based can in general be such that it has both healthy or nonmalignant cells with intact EGF receptor and the associated encoding wild type genes, as well as tumor cells with optionally mutated EGF receptor and the associated relevant mutation genes or mutation alleles. For example, the sample can be one that also has circulating tumor cells, along with intact cells.

Before the method according to the present invention is carried out, the sample or the starting material can also be further purified or the DNA from the sample can be isolated and/or concentrated and/or purified for further use. The relevant methods are sufficiently familiar to a person skilled in the art, so no further description is necessary.

In the context of the present invention it is possible that gene modifications or mutations can be determined on the basis of a sample that has less than 1000, for example, less than 500, for example, less than 200, for example, less than 100 tumor cells per ml of sample. The method according to the present invention can be carried out on the basis of samples or starting materials with DNA, which have less than 1%, for example, less than 0.1%, for example, less than 0.01%, for example, less than 0.001% tumor cells, relative to the total cell content of the sample.

The method according to the present invention can be applied with respect to a sample or a starting material that has less than 1% mutated DNA, for example, about 0.5%, for example, about 0.05%, for example, about 0.005%, for example, about 0.0005% mutated DNA, relative to the total DNA in the sample or in the starting material. The method according to the present invention is therefore a highly sensitive and efficient method in which even minute traces of DNA can be analyzed effectively with respect to the presence of mutations.

An analysis or evaluation is presented in more detail below, purely as a nonlimiting example, based on a test conducted with a parallel wild type reference assay.

Regarding the analysis of the melting curves, in general, the melting curve with the lowest specific melting temperature or the highest melting temperature range is assigned to the mutation gene, i.e., the presence of different melting curves with different melting points or ranges can be taken as evidence or an indication of the presence of a mutation, wherein the curve with the higher melting point or melting range is to be attributed to the firmer binding of the sensor probe to the wild type single-stranded DNA and therefore to the unmutated form. If several melting curves are present, the melting curve with the lower specific melting temperature or the lower melting temperature range can moreover be assigned to the mutation gene, wherein the associated melting curve with the lower melting temperature or the lower melting temperature range is caused by the less pronounced and nonspecific binding of the sensor probe to the mutated segment of the wild type single-stranded DNA. If wild type DNA is also present in the sample to be analyzed, a melting point or range occurring at higher temperatures can equally develop for the melting curve of the genetically modified or mutated form, which owing to the measures carried out according to the present invention generally, however, has a smaller signal maximum (e.g., suppression of binding of the sensor probe and/or suppression of amplification).

In the context of the present invention, apart from the aforementioned determination or analysis of whether a mutation is present in a sample and what mutation it is, it is moreover equally possible to find information about whether the mutation is a homozygotic or heterozygotic gene modification. With respect to the sample, it is to be assumed that the assay containing the mutation gene to be analyzed is at least substantially not contaminated with wild type genes. In the context of the method according to the present invention, it can thus be envisaged that the melting curve with the lowest melting point and/or the lowest melting range is assigned to a homozygotic gene modification for the case when the melting curve has a single melting point or melting range. In the context of the method according to the present invention, the procedure adopted can moreover be that the melting curve with the lowest melting point or the lowest melting range is assigned to a heterozygotic gene modification for the case when the melting curve has two melting points or melting ranges different from one another.

For detecting homozygotic or heterozygotic gene modifications in a sample that also contains the wild type gene, the procedure adopted can be, for example, that a first PCR assay of the sample without a blocking agent and a second parallel PCR assay with a blocking agent are carried out and analyzed, optionally with another reference assay based on the wild type gene. By comparing the formation of the melting points or ranges, in particular, with respect to the corresponding signal maxima of the wild type genes or wild type alleles in the samples with or without blocking agent, it can be analyzed for the presence of a homozygotic or heterozygotic form of mutation.

Based on the specific combination of all measures according to the present invention, in the context of the present invention, it is in particular possible to have an at least 10-fold, for example, at least 50-fold, for example, at least 100-fold, for example, at least 500-fold, for example, at least 1000-fold intensification or amplification of detection with respect to the gene segment having the gene modification, for example, DNA segment, of the mutation gene or of the concomitant (fluorescence) signal. In the context of the present invention, it is possible to have an at least 10-fold, for example, at least 50-fold, for example, at least 100-fold, for example, at least 500-fold, for example, at least 1000-fold intensification and/or amplification of the measurement signal associated with the gene segment having the gene modification, for example, DNA segment, of the mutation gene.

As a result, in the context of the present invention, it was possible, based on the combination of the aforementioned measures, to provide an exceptionally sensitive detection of mutations. In the context of the present invention, a mutation can be analyzed or detected even if only very small amounts of mutated DNA are present in the sample.

The method according to the present invention is equally suitable for monitoring progression based on the underlying body material of the sample, for example, peripheral blood, which provides simple monitoring of the course of the disease at the molecular level. On the basis of the method according to the present invention, optionally in combination with further purification methods, which, in particular, are based on specific purification or isolation of the DNA to be investigated from the sample and which are familiar as such to a person skilled in the art, in the context of the present invention, it is possible to detect circulating tumor cells in whole blood.

The method according to the present invention is therefore suitable for finding information for the diagnosis of the tumor or cancer disease or for determining the risk of developing a tumor or cancer disease, or for prognosis of the course of a tumor or cancer disease or for prognosis of individual drug effects during treatment of the tumor and/or cancer disease. For example, and non-limitatively, a patient or test subject can be assigned an increased risk of developing a bronchial carcinoma if he/she has one or more of the aforementioned mutations, which can be detected on the basis of the method according to the present invention. A disease prognosis or a disease course can also be recorded or analyzed, wherein in this connection, samples can be taken from the patient or test subject over a defined period of time and can be analyzed on the basis of the method according to the present invention and detection of the corresponding mutations can be taken as an indication of the presence of tumor cells in the sample.

In the context of the present invention, it is also possible, based on the concrete analysis of the underlying mutation, to optimize the therapeutic approach, in particular, with respect to the specific medication. Thus, based on the mutations found, the respective specific drugs or those with optimum effects can be used, which leads to further personalization and specification of medication or therapy, accompanied by greater therapeutic efficacy. If the ΔE746-A750 mutation and/or the T790M mutation are thus present, optionally in combination with the L858R mutation, application or administration of the second-generation inhibitors described above may be indicated, whereas if the L858R mutation is present, administration of the first-generation inhibitors described above may be indicated.

As mentioned above, the method according to the present invention can be used for mutation analysis in the case of bronchial carcinomas and, in particular, non-small cell lung cancer or NSCLC with the associated involvement of the EGF receptor.

In an embodiment, the present invention provides a method of detecting a gene modification, in particular, mutation, in a gene coding for the EGF receptor, wherein the EGF receptor is, in particular, associated with a tumor or cancer disease, in particular, a lung carcinoma, such as non-small cell lung cancer (NSCLC), in particular, wherein the gene that has the gene modification (mutation gene) is present together with other genes coding for the protein, but not having a gene modification (wild type genes). The method according to this aspect of the present invention is also characterized in that, in particular, by means of an asymmetric polymerase chain reaction (PCR) with combined use of at least one detectable wild type-specific hybridization probe, only binding nonspecifically to the mutation gene (sensor probe) on the one hand, and optionally at least one wild type-specific blocking agent inhibiting the binding of the sensor probe to the wild type gene and optionally an anchor probe on the other hand, is carried out, in particular, so that there is selective intensification and/or amplification of detection with respect to a gene segment having the gene modification, in particular, DNA segment, of the mutation gene.

The methods according to the present invention, according to the above aspects, can be carried out as such based on methods known per se by a person skilled in the art employing the relevant apparatus and measuring instruments. For example, the polymerase chain reaction and recording and analysis of the corresponding melting curves can be carried out using a LightCycler®-480 apparatus from the company F. Hoffmann-La Roche Ltd.

In an embodiment, the present invention provides a composition, for example, for use in the context of an asymmetric polymerase chain reaction (PCR), for example, for detecting at least one gene modification, such as a mutation in a gene, for example, in a gene that codes for a protein associated with a tumor and/or cancer disease, for example, wherein the gene that has the gene modification (mutation gene) is present together with further genes coding for the protein, but not having a gene modification (wild type genes), wherein the composition contains in combination:
a detectable wild type-specific hybridization probe (sensor probe), in particular, as defined above;
a first primer, which binds at least substantially specifically to the single-stranded DNA of the mutation gene (mt-probe strand), with which the sensor probe is capable of interacting;
a second primer, which is capable of interacting at least substantially specifically with the DNA single strand of the mutation gene complementary to the probe strand (mt-complementary strand);
a wild type-specific blocking agent inhibiting the binding of the sensor probe to the wild type gene;

wherein the content and/or the amount of the first primer (b) in the composition is greater than the content and/or the amount of the second primer (c).

Regarding the composition according to the present invention, it can contain (a) the hybridization probe or sensor probe in an amount from 0.01 to 5 pmol/μl for example, 0.05 to 3 pmol/μl, for example, 0.1 to 1 pmol/μl, relative to the composition.

The composition (b) according to the present invention can moreover contain the first primer at a concentration of 0.05 to 10 pmol/μl, for example, 0.1 to 5 pmol/μl, for example, 0.2 to 2 pmol/μl, relative to the composition.

The composition (c) according to the present invention can furthermore contain the second primer at a concentration of 0.005 to 5 pmol/μl, for example, 0.01 to 2 pmol/μl, for example, 0.03 to 0.5 pmol/μl, relative to the composition.

The composition according to the present invention should contain (a) the first primer and (b) the second primer in a quantitative ratio of (a) the first primer to (b) the second primer ((a): (b)), relative to the composition, in the range from 1000:1 to 1.05:1, for example, 100:1 to 1.5:1, for example, 10:1 to 2:1.

The composition can moreover contain (d) the blocking agent at a concentration of 0.005 to 4 pmol/μl, for example, 0.01 to 1 pmol/μl, for example, 0.015 to 0.1 pmol/μl, relative to the total volume of the composition.

The composition according to the present invention can, in particular, contain (e) an anchor probe, in particular, as defined above, for example, in an amount from 0.01 to 5 pmol/μl, for example, 0.05 to 3 pmol/μl, for example, 0.1 to 1 pmol/μl, relative to the composition.

The composition is, in particular, an aqueous solution or dispersion. In this connection the composition can, in particular, contain PCR-pure water. The composition can moreover contain a so-called 480-probes-master.

The present composition can be prepared or portioned ready for use and can be cooled or frozen for keeping or storage.

In the context of the present invention, it is also possible that the composition is at least partially in the form of spatially separated components, in particular, as a kit-of-parts, wherein the components (a) to (d) and optionally (e) can be present at least partially separated from one another. In this connection, the components or constituents can, for example, be brought together immediately before carrying out the test to obtain a composition that is ready for use.

According to a third aspect of the present invention, the present invention relates to the use of the composition as defined above for detecting at least one gene modification, in particular, a mutation, in a gene, for example, in a gene coding for a protein, that is associated with a tumor and/or cancer disease, in particular, wherein the gene that has the gene modification (mutation gene) is present together with further genes coding for the protein, but not having a gene modification (wild type genes), in particular, in the context of an asymmetric polymerase chain reaction.

The present invention is illustrated further in the drawings, purely as examples, but without limiting the present invention.

FIG. 1 shows a schematic representation of the exon distribution of the human EGF receptor gene on the functional domains in the corresponding protein and an overview of the mutations associated with the development or the presence of drug resistance or drug sensitivity (cf. Sharma et al. “Epidermal growth factor receptor mutations in lung”; Nature Reviews, 2007, 7: 169-181). The designation “EGF binding” relates to the region of the ligand binding site in the corresponding protein, “TM” to the transmembrane domain, “nucleotide-binding loop” to the nucleotide-binding region and “activation loop” to the activation region. Some of the mutations represented are associated with drug resistance with respect to the treatment of lung cancer, whereas other mutations are associated with a change in drug sensitivity. About 45% of all known mutations of the EGF receptor that are associated with non-small cell lung cancer are localized in the region of exon 19. The mutations presented in FIG. 1 for exon 19 of the EGF receptor are deletions centering on amino acid 746 or 747.

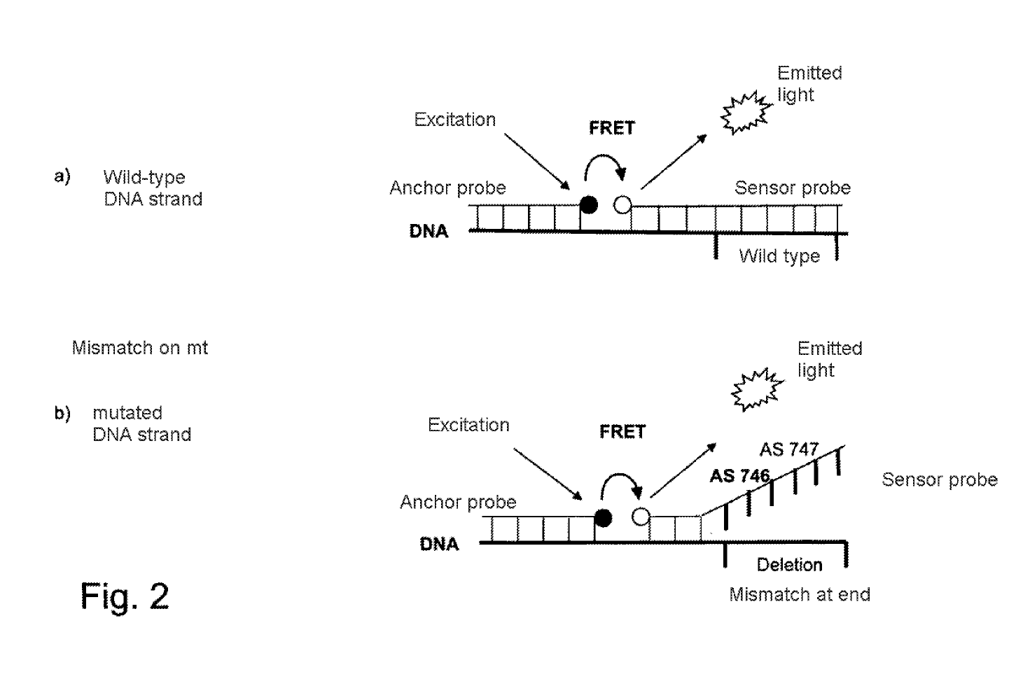

FIG. 2 explains the principle of detection according to the present invention using special hybridization probes. In this connection, FIG. 2 a) shows the binding of the respective hybridization probes, namely sensor probe on the one hand and anchor probe on the other hand, to a single strand of DNA without gene modification and therefore to the wild type. The sensor probe here binds specifically in the region whose corresponding region in the mutation gene contains the mutation, in particular, the deletion. Through excitation of the fluorescent dye of the sensor probe and transfer of the energy by FRET onto the dye of the anchor probe, a signal is emitted which can be detected or measured. So that energy transfer can take place, it is of particular advantage according to the present invention if the base spacing between the probes is not more than 5 bp. The fluorescent dye of the sensor probe is excited by light, the energy is transferred by FRET (fluorescence-resonance energy transfer) to the fluorescent dye of the anchor probe, and the emitted light can be measured. FIG. 2 b) shows the binding of the two hybridization probes to a single strand of DNA with gene modification or mutation, in particular, in the form of a deletion. As the sensor probe binds specifically to the wild type strand or is complementary to the wild type strand, in this case it can only hybridize to the mutated strand or mutation strand incompletely or only in sections or nonspecifically. Also in this case, if binding is present, FRET can take place first. Because the binding of the sensor probe to the wild type strand is incomplete or only in sections or nonspecific, on heating, there is earlier detachment of the sensor probe from the mutation strand compared with the wild type strand, so that the FRET signal with respect to the mutation strand decreases or disappears (not shown).

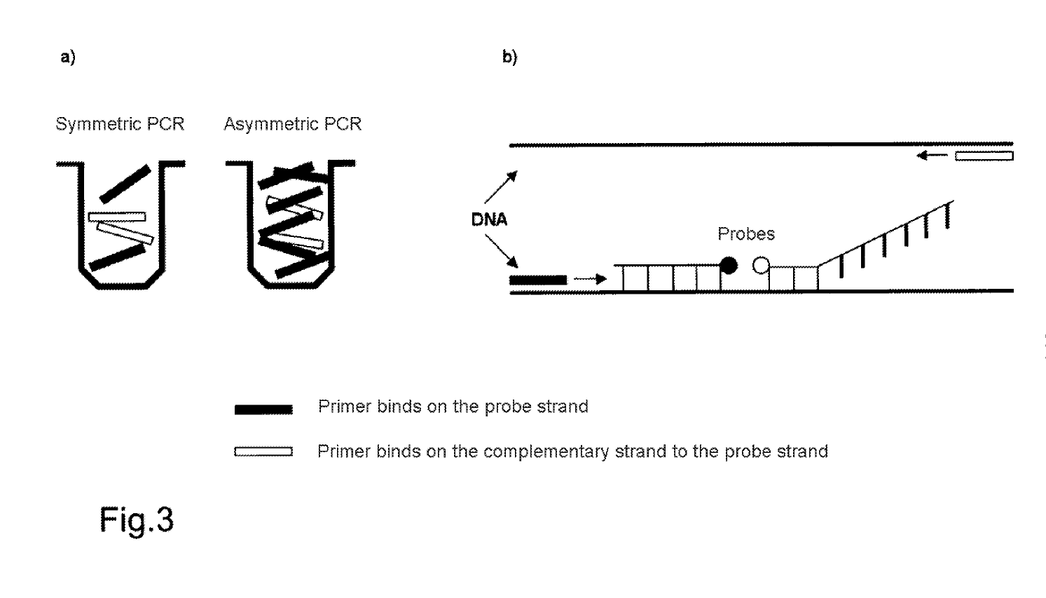

FIG. 3 explains the principle of the asymmetric PCR carried out according to the present invention. FIG. 3 a) shows that, in contrast to symmetric PCR, the primer ratio is not identical in asymmetric PCR. The concentration of the primer that binds to the probe strand is increased (shown in black here). FIG. 3 b) shows an overview according to which the primer shown in black binds to the probe strand, whereas the primer shown in white binds to the complementary single-stranded DNA.

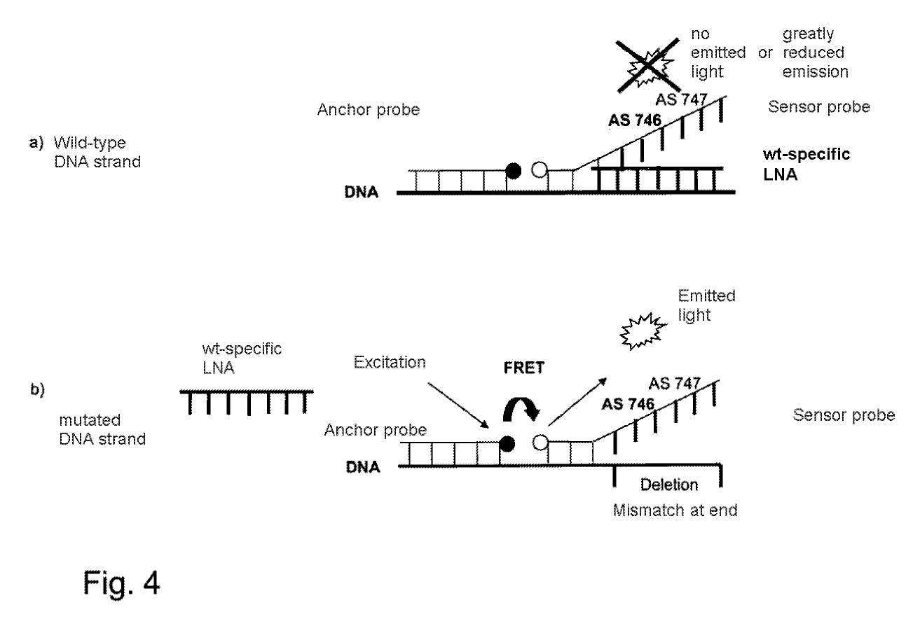

FIG. 4 illustrates the principle of inhibition of the wild type single-stranded DNA by the blocking agent used according to the present invention. FIG. 4 a) shows the binding of the specific blocking agent for the wild type (wt) to the wild type single-stranded DNA. At this point, the sensor probe can, therefore, at least substantially no longer hybridize. The wt-DNA is no longer amplified and also, in the absence of FRET, does not emit a signal. FIG. 4 b) shows the situation in which the blocking agent cannot bind on the mutated single-stranded DNA, but the sensor probe can. The mutated DNA is amplified and emits a signal, in particular, in the form of light, which can be measured.

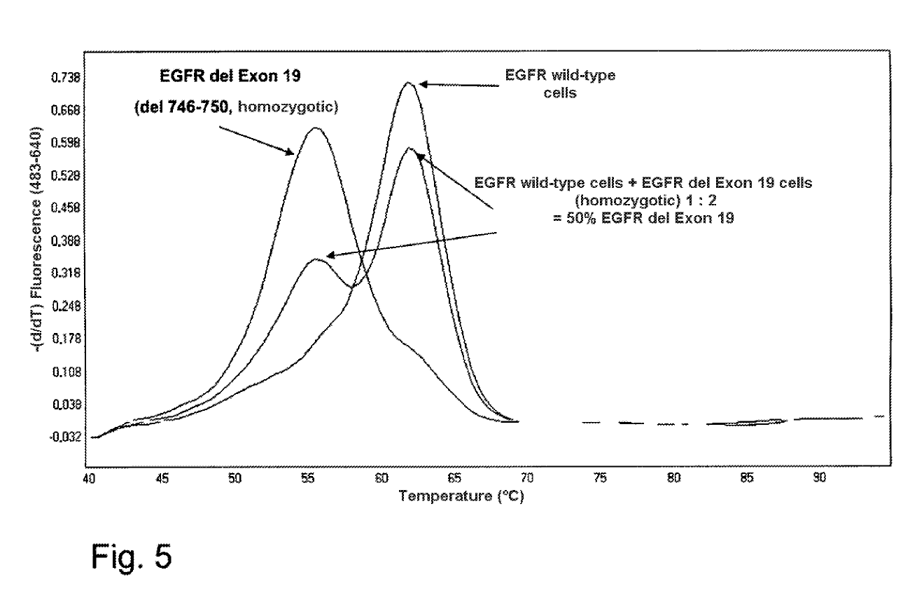

FIG. 5 presents specific melting curves based on a homozygotic exon 19_del_{—}746-750 mutation, a 1:2 mixture of the homozygotic exon 1_del_{—}746-750 mutation and of the wild type using a wild type-specific sensor probe and without adding a blocking agent. The actual PCR is followed by a melting curve analysis.

The decrease in fluorescence, caused by the detachment of the FRET probes from the DNA strand owing to the progressive temperature increase, is measured as a function of temperature. For better visualization, the first mathematical derivative of the curve of intersection is calculated so that the turning point of the measured fluorescence is shown as a curve. The highest point or the maximum of the curve corresponds to the specific melting temperature. The curve with the maximum at about 65° C. shows a wild type sample whose melting point, owing to the wild type-specific sensor probe, is increased relative to the mutant. The curve with the maximum at about 55° C. shows the exon 19_del_{—}746-750 mutation sample, which has a lower melting point compared to wild type cells. The 1:2 mixture of wild type and exon 19_del_{—}746-750 mutation shows, similar to a heterozygotic mutant, a “double peak” or two melting points, as it has both the melting point or the melting point range of the wild type and of the exon 19_del_{—}746-750 mutation.

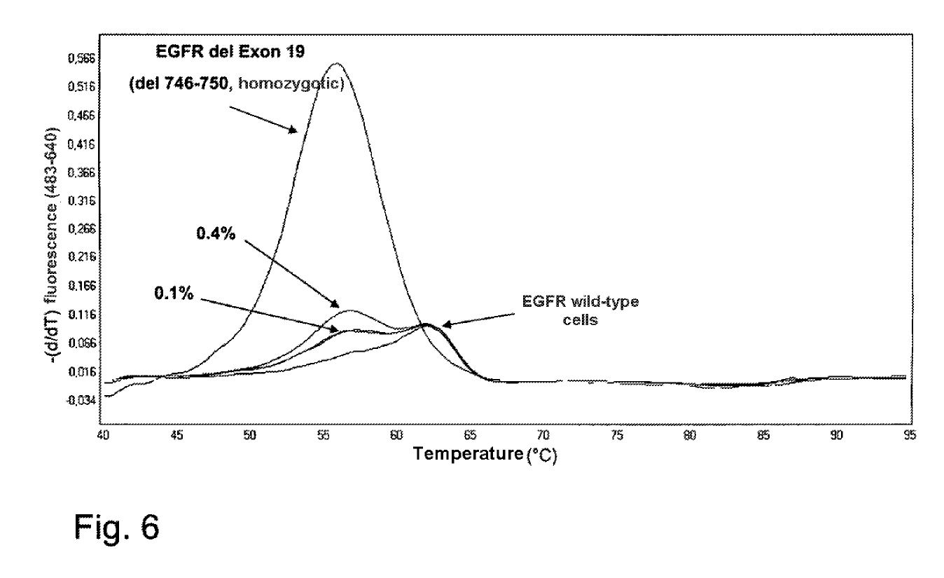

FIG. 6 presents the specific melting curves of a homozygotic exon 19_del_{—}746-750 mutation and its 0.1% and 0.4% dilutions and of the wild type, in each case with addition of a wild type-specific blocking agent, which prevents or at least reduces the binding of the sensor probe to the wild type probe strand of the EGF receptor gene. The actual PCR is followed by a melting curve analysis. The decrease in fluorescence, caused by the detachment of the FRET probes from the DNA strand owing to the progressive temperature increase, is measured as a function of the temperature. For better visualization, the first mathematical derivative of the curve of intersection is calculated so that the turning point of the measured fluorescence is shown as a curve. The highest point or the maximum of the curve corresponds to the specific melting temperature. The curves with the maximum at about 55° C. show the exon 19 del_{—}746-750-mutation sample and dilutions thereof. By carrying out the PCR with addition of a blocking agent, the wild type signal is largely suppressed. In this way, effective detection of mutations is possible even with considerable dilution, as occurs for example in a biological sample or a sample from a test subject.

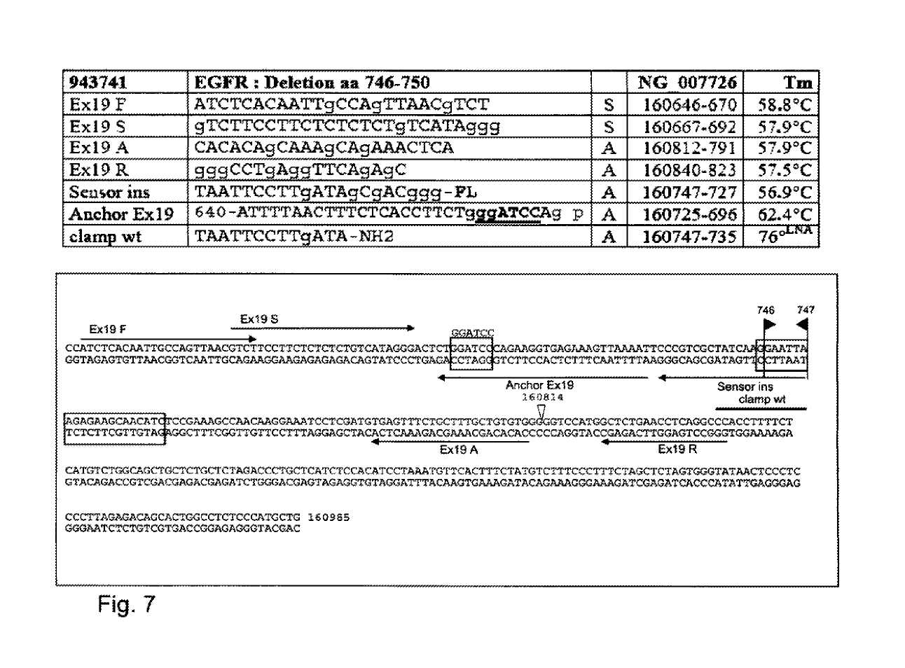

FIG. 7 shows an overview of primers that can be used according to the present invention (Ex19F, Ex19S, Ex19A, Ex19R), probes (Sensor ins, Anchor Ex19) and of the wild type-specific blocking agent (clamp wt) and a schematic representation of the binding sites of the primer pairs used or tested and of the probes used. These primers and hybridization probes are obtainable, for example, from TIB MOLBIOL GmbH, Berlin, Germany. The binding sites of the primers Ex19F, Ex19S, Ex19A and Ex19R are characterized, for example, a combination of Ex19S and Ex19R, and of the sensor probe Sensor ins and the anchor probe Anchor Ex19, on the DNA sequence of the EGF-gene.

In connection with the present invention, it was shown that the activation of the EGF receptor (EGFR) can be inhibited by EGFR inhibitors. Over 80% of the mutations of the EGF receptor in NSCLC patients are based on various deletions in exon 19, such as the deletion ΔE746-A750, and on a point mutation in exon 20, namely L858R (substitution of the amino acid leucine L in position 858 with arginine R, cf. FIG. 1). Patients with a lung tumor, who have one of these changes, are therefore particularly suitable for treatment with EGFR inhibitors.

Although the therapy is generally well tolerated and is very specifically effective, most patients develop a so-called secondary mutation after a certain time, which can develop in addition to the mutation already present and leads to resistance to erlotinib and gefitinib. The mutation T790M (substitution of the amino acid threonine T with methionine M in position 790, cf. FIG. 1) is found in about 50% to 65% of these cases. For these patients, drugs are available whose mechanism of action is different from that of the first-generation drugs (erlotinib, gefitinib). These so-called second-generation inhibitors bind irreversibly to the receptor and not reversibly, such as for example Tarceva® or Iressa™. NSCLC patients who, because of the T790M mutation, no longer respond to first-generation drugs, can therefore be treated further with a second-generation EGFR inhibitor (e.g., BIBW2992/Tovok from Boehringer Ingelheim). As shown in clinical studies, these inhibitors are also highly specific and effective, so that the growth and survival of the tumor cells can be slowed down or prevented.

In the sense of personalized medicine, i.e., to be able to offer each individual patient with NSCLC the therapy that is most suitable for him, it is necessary and sensible to test the tumor tissue for the EGFR status. Those patients who have an activating mutation in the EGF receptor can then be offered treatment with the corresponding EGFR inhibitors.

In order to achieve a high degree of sensitivity, in the context of the present invention, various optimization steps are performed, which will be illustrated in detail below for the example of the deletion ΔE746-A750.

At the beginning of the planning or execution of mutation detection, specific primers and hybridization probes are first generated or provided. These primers and hybridization probes can be obtained for example from TIB MOLBIOL GmbH, Berlin.

The DNA segment in which the mutation to be detected is localized is, for example, amplified by the specific primers. In addition, according to an embodiment of the present invention, two hybridization probes that are different from one another (sensor probe on the one hand and anchor probe on the other hand) are used in the PCR reaction. These comprise, as do the primers, several nucleotides, which hybridize within the resultant PCR product. The two probes moreover bind in close vicinity to one another. At the ends opposite one another, the probes are each labeled with a fluorescent dye and can interact with one another by FRET (fluorescence-resonance energy transfer) (cf. FIG. 2). The probes bind both in and on the wild type sequence (wild type single-stranded DNA or wild type DNA strand) and on the mutation sequence (mutated single-stranded DNA or mutated DNA strand), wherein one of the probes (anchor probe) binds in the unaltered region of the sequence and the other probe (sensor probe) with respect to the wild type DNA, binds in the region corresponding to the presumed mutation, whereas with respect to the mutation as such on the mutated DNA, there is at least substantially no binding, so that, with respect to the mutated DNA, there is only binding of the sensor probe in sections, namely on a region of the mutated DNA directly adjoining the mutation site. As a result, binding of the sensor probe to the mutated DNA therefore only occurs in sections. As mentioned above, the sensor probe binds on the unmutated and therefore wild type DNA specifically.

In both cases, during or after energetic excitation with electromagnetic radiation of a corresponding wavelength, such as can be produced by the xenon lamp of the LightCycler®, the sensor probe transfers its energy by FRET to the anchor probe. This is also excited as a result and emits a fluorescence signal, which can be detected by the equipment. Measurement can be carried out at the end of the annealing phase of each PCR cycle, the phase in which the primers and probes bind to the DNA strand.

The actual PCR is followed by a melting curve analysis. The reaction mixtures are here heated slowly, for example, to 95° C. At the point where 50% of the sensor probes have detached from the resultant PCR products, there is a dramatic drop in fluorescence. In the case of specific binding of the probe to the unmutated strand, the dehybridization or detachment of the sensor probe takes place at a higher temperature compared to binding to mutation sequences. In addition to the decrease of the fluorescence signal with increasing temperature, in particular, using the LightCycler® 480, the first mathematical derivative of the curve of intersection is calculated, so that the turning point of the measured decrease in fluorescence is represented as a curve. The highest point or the maximum of the curve based on the first derivative corresponds to the specific melting temperature. This representation serves for better visual evaluation. Samples that possess both a wild type and a mutated allele therefore show two different melting profiles, which can be seen correspondingly as a “double peak” or double maximum (formation of two different maxima) (cf. FIG. 5).

After possible optimization of the amount of DNA used, of the annealing temperature, of primer compatibility and/or concentration and of probe concentration, detection of the mutation in the conditions of the prior art, in the context of a usual and non-asymmetric PCR, just as in the case of sequencing, shows a sensitivity of only 20 to 25%.

In order to increase the limit of detection in the context of the present invention in an appropriate way, a so-called asymmetric PCR is applied or carried out. In contrast to symmetric PCR, in this case, the ratio of the two primers to one another is not equal. The concentration of the primer that binds to the DNA strand to which the probes also hybridize (probe strand) is increased in the PCR mixture. Therefore, this strand is, for example, multiplied and the probes used therefore have a higher probability of binding to this strand (cf. FIG. 3). In this way, the sensitivity of mutation detection can also be increased.

To increase the sensitivity further according to the present invention, it is also envisaged to reduce or inhibit the amplification of the wild type DNA strand and/or the binding of the sensor probe to the wild type DNA strand so that only or primarily the mutated DNA strand is multiplied and the probes used therefore can as it were interact with the mutated DNA without competition. Surprisingly, by means of this technology, even minute amounts of mutated material can be detected. This specific inhibition of the wild type can be carried out, for example, with a blocking agent, in particular, in the form of a so-called locked nucleic acid (LNA™ from the company Exiqon), which is also called “clamp” (cf. FIG. 4). These “clamps” are modified biochemically so that they have a very high affinity for the wild type strand and only melt at very high temperatures.

During the melting curve analysis, the decrease in fluorescence is measured. If a mutation is to be detected in the sample, the corresponding curve is visible; if no mutated material is present, there is no or only a minimal wild type curve to be seen, because owing to the absence of amplification and probe binding of the wild type, no signal can be measured (cf. FIG. 6).

To be able to confirm that the PCR reaction has been carried out successfully and in order to be able to detect heterozygotes, a comparative assay without blocking agent should be conducted in parallel or carried out. In this reaction, at least the wild type should be visible.

The combination according to the present invention of the aforementioned measures permits mutation detection with extremely high sensitivity. In the aforementioned example of the detection of the ΔE746-A750 mutation, a sensitivity of 0.0005% of mutated DNA in a wild type mixture can be achieved. Minute proportions of mutated DNA, for example, of a tumor tissue, can therefore be detected in a sample. The method according to the present invention is also suitable for monitoring progression in the peripheral blood or other body fluids. In many patients with solid tumors, for example, NSCLC patients, mostly tumor material is obtained only once, e.g., at the first diagnosis or during an operation. Monitoring or observation of the course of the disease at the molecular level is therefore not generally possible. With the highly sensitive method of detection according to the present invention, which can also be carried out in combination with sensitive purification methods, even circulating tumor cells in a whole blood sample can be detected. For example, it has been described that patients with NSCLC have about 100 to 200 circulating tumor cells per 1 ml of blood. If we assume about 10,000 nucleated cells per μl, that means a content of about 0.001% of tumor cells. With the real-time PCR method according to the present invention, which can also comprise a sensitive method of purification of the tumor cells, detection and therefore monitoring of progression in patients is possible before or during therapy. The present invention therefore represents an important step in the direction of personalized and therefore highly specific therapy.

Further configurations, modifications and variations of the present invention can readily be recognized and can be carried out by a person skilled in the art on reading the description, while remaining within the scope of the present invention.

The following practical example only serves to illustrate the present invention, but without restricting the present invention thereto.

### Example

### EGFR Deletion Exon 19 mutation Detection on the LC480

Detection of various deletions in exon 19 of the EGF receptor in genomic DNA by means of melting curve analysis on the Light Cycler 480.

### Additional Reference Documents

Data sheet “LightCycler 480 Probes Master” of F. Hoffmann-La Roche Ltd.

Data sheet “PureLink Genomic DNA Mini Kit” of Invitrogen Corp.

### Material

LightCycler® 480 Probes Master Kit of F. Hoffmann-La Roche Ltd. (Order No. 04887301001).

Primer and probes from TIB MOLBIOL GmbH, Berlin.

Primer: Ex 19 S (Prod. No. 1126164, provisional Sequence Listing II, SEQ ID NO. 2) and Ex 19 R (Prod. No. 1126166, provisional Sequence Listing II, SEQ ID NO. 3).

Probes: Anchor Ex 19 (Prod. No. 1131500, provisional Sequence Listing II, SEQ ID NO. 5) and Sensor ins (wild type-specific sensor probe, provisional Sequence Listing II, SEQ ID NO. 4) and LNA: clamp wt (Prod. No. 1137307, provisional Sequence Listing II, SEQ ID NO. 6).

Genomic DNA, e.g. from peripheral blood (citrate).

(20-90 μg/ml initial concentration).

Isolated with “PureLink Genomic DNA Mini Kit” from Invitrogen Corp. (Order No. K1820-01).

Control DNA: e.g. A431 (EGFR wild type) and HCC-827 (EGFR deletion in exon 19).

DNA vectors: plasmid MS658 (pcDNA3.1/V5-His-TOPO-EGFR_Exon19_Del. 746-750).

96-well plates from F. Hoffmann-La Roche Ltd., white (LightCycler 480-Multiwell-Plate 96, Order No. 04729692001).

### Equipment

centrifuge with insert for 96-well plates (e.g. Multifuge 3SR+ from Thermo Fisher Scientific Inc.).

LightCycler® 480 from F. Hoffmann-La Roche Ltd. with 96-well-block.

### Description of Procedure

Thawing of the required kit components, primers and probes on ice (thaw probes and 480-probes-master in the dark).

Pipetting of the primer/probes mixes on ice (without LAN):

| | | |
|---|---|---|
| Ex 19 S: | 0.10 μl (2 pmol) | 20 pmol/μl |
| + Ex 19 R: | 0.10 μl (2 pmol) | 20 pmol/μl |
| + Anchor Ex 19: | 0.15 μl (3 pmol) | 20 pmol/μl |
| + Sensor ins: | 0.15 μl (3 pmol) | 20 pmol/μl |
| + PCR-pure water: | 1.50 μl | |
| Total volume: | 2 μl | |

Pipetting of the primer/probes mixes on ice (with LNA):

| | | | |
|---|---|---|---|
| Ex 19 S: | 0.10 μl | (2 pmol) | 20 pmol/μl |
| + Ex 19 R: | 0.10 μl | (2 pmol) | 20 pmol/μl |
| + Anchor Ex 19: | 0.15 μl | (3 pmol) | 20 pmol/μl |
| + Sensor ins: | 0.15 μl | (3 pmol) | 20 pmol/μl |
| + Clamp wt: | 0.30 μl | (6 pmol) | 20 pmol/μl |
| + PCR-pure water: | 1.20 μl | | |
| Total volume: | 2 μl | | |

Pipetting of the master mix:

| | |
|---|---|
| Primer/probes mix according to above details: | 2 μl |
| + 480-probes master: | 10 μl |
| + PCR-pure water: | 7.5 μl |
| Total volume: | 19.5 μl |

19.5 μl master mix and 0.5 ng DNA in 0.5-8 μl total volume are pipetted into a white 96-well plate. If the DNA is dissolved in more than 0.5 μl, the PCR-pure water from point 4 is reduced correspondingly. As individual values, the following controls are always conducted at the same time, in each case, once with LNA and once without LNA: water control without DNA, negative control (wild type, e.g. A431), positive control (mutant, e.g., HCC-827) and a second positive control (plasmid MS658).

Patient samples are analyzed at the same time as individual value without LNA, as double value with LNA.

Sealing of the plate with film and centrifugation: 1500 g, 2 min at RT.

Put the plate in LightCycler® 480 and start program:

| Filter | Melt | Quantum | Max. integration |
|---|---|---|---|
| combinations: | factor: | factor: | time: |
| 483-640 | 1.2 | 5 | 2 seconds |
| Protocol: | Temperature | Time | Cycles |
| 1. Preincubation: | 95° C. | 00:10:00 | 1 |
| 2. Amplification: | 95° C. | 00:00:10 | 40 |
| | 60° C. | 00:00:10 | |
| | 72° C. | 00:00:10 | |
| 3. Melting curve: | 95° C. | 00:01:00 | 1 |
| | 40° C. | 00:02:00 | |
| | 95° C. | continuous | |
| 4. Cooling | 4° C. | continuous | |

Expected result of the melting curve analysis:

| Result: | Melting curve temperatures |
|---|---|
| Probes bind specifically | |
| to wild type | |
| homozygotic mutant | one melting curve |
| (HCC 827): | between 45 and 60° C. |
| homozygotic mutant | one melting curve |
| (MS 658) | between 47 and 55° C. |
| homozygotic | one melting curve |
| wild type: | between 55 and 70° C. |
| heterozygosity: | two melting curves; see above |

Instructions and notes: The prepared PCR-mix is stable for at least 24 hours at RT in the dark.

Abbreviation: RT=room temperature. Moreover, generally

The present invention is not limited to embodiments described herein; reference should be had to the appended claims.

What is claimed is:

1-36. (canceled)

37. A method for detecting at least one gene modification, such as a mutation in a gene, such as a gene that codes for a protein associated with at least one of a tumor and a cancer, the method comprising:
1) providing a detectable hybridization probe (sensor probe) which interacts with/binds to a gene not having a gene modification (wild type gene) and with a gene having a gene modification (mutation gene), wherein the detectable hybridization probe (sensor probe) has at least one of a higher specificity, a higher binding affinity and a higher selectivity for the gene not having a gene modification (wild type gene) compared to the gene having a gene modification (mutation gene); and
2) detecting at least one gene modification with the detectable hybridization probe (sensor probe).

38. The method as recited in claim 37, wherein the at least one gene modification is at least one of a frameshift mutation, a deletion, an insertion, and a point mutation.

39. The method as recited in claim 37, wherein the detectable hybridization probe (sensor probe) can interact with/bind to a single-stranded DNA (wt-probe strand) of the gene not having a gene modification (wild type gene) and to a corresponding single-stranded DNA having the gene modification (mt-probe strand) of the gene having a gene modification (mutation gene),
wherein the detectable hybridization probe (sensor probe) has at least one of a higher specificity, a higher binding affinity and a higher selectivity for the single-stranded DNA (wt-probe strand) of the gene not having a gene modification (wild type gene) compared to the corresponding single-stranded DNA having the gene modification (mt-probe strand) of the gene having a gene modification (mutation gene).

40. The method as recited in claim 39, wherein the detectable hybridization probe (sensor probe) has at least one of a lower specificity, a lower binding affinity and a lower selectivity for the single-stranded DNA (mt-probe strand) of the gene having a gene modification (mutation gene) compared to the corresponding single-stranded DNA (wt-probe strand) of the gene not having a gene modification (wild type gene).

41. The method as recited in claim 39, wherein the detectable hybridization probe (sensor probe) is at least one of specifically bindable with and completely hybridizable to the single-stranded DNA (wt-probe strand) of the gene not having a gene modification (wild type gene).

42. The method as recited in claim 39, wherein the detectable hybridization probe (sensor probe) is interactable with/bindable to the single-stranded DNA (wt-probe strand) of the gene not having a gene modification (wild type gene) over a complete nucleotide sequence at least one of substantially completely and at least substantially.

43. The method as recited in claim 39, wherein the detectable hybridization probe (sensor probe) is interactable with/bindable to a segment of the single-stranded DNA (wt-probe strand) of the gene not having a gene modification (wild type gene) corresponding to a segment of the at least one gene modification of the single-stranded DNA (mt-probe strand) of the gene having a gene modification (mutation gene) over a complete nucleotide sequence at least one of substantially completely and at least substantially.

44. The method as recited in claim 39, wherein the detectable hybridization probe (sensor probe) has an at least substantially complementary nucleotide sequence to the single-stranded DNA (wt-probe strand) of the gene not having a gene modification (wild type gene).

45. The method as recited in claim 39, wherein the detectable hybridization probe (sensor probe) at least one of nonspecifically binds to, incompletely binds to, and sectionally binds with the single-stranded DNA (mt-probe strand) of the gene having a gene modification (mutation gene).

46. The method as recited in claim 39, wherein the detectable hybridization probe (sensor probe) is at least one of substantially not capable of binding to and not capable of interacting with the single-stranded DNA (mt-probe strand) of the gene having a gene modification (mutation gene) at at least one of a position of and at a site of the gene modification.

47. The method as recited in claim 39, wherein the detectable hybridization probe (sensor probe) cannot interact with/bind to the single-stranded DNA (mt-probe strand) of the gene having a gene modification (mutation gene) at at least one of a position of and at a site of the gene modification.

48. The method as recited in claim 39, wherein the detectable hybridization probe (sensor probe) only interacts with/binds to the single-stranded DNA (mt-probe strand) of the gene having a gene modification (mutation gene) in sections of the nucleotide sequence of the detectable hybridization probe (sensor probe) such as an edge segment, a marginal region, an end segment, and a terminal region.

49. The method as recited in claim 39, wherein the detectable hybridization probe (sensor probe) interacts with/binds to at least one of a segment and a region of the single-stranded DNA (mt-probe strand) of the gene having a gene modification (mutation gene) following at least one of a position and a site of the gene modification.

50. The method as recited in claim 39, wherein the detectable hybridization probe (sensor probe) has at least one binding region/section and at least one non-binding region/section compared to the single-stranded DNA (mt-probe strand) of the gene having a gene modification (mutation gene).

51. The method as recited in claim 50, wherein a number of nucleotides of the at least one non-binding region/section corresponds at least substantially to a number of nucleotides forming the at least one gene modification.

52. The method as recited in claim 50, wherein a number of nucleotides of the at least one binding region is from 1 to 30.

53. The method as recited in claim 37, wherein the detectable hybridization probe (sensor probe) has at least one of at least 3 nucleotides and a number of nucleotides from 3 to 60.

54. The method as recited in claim 39, wherein the detectable hybridization probe (sensor probe) has a ratio of binding nucleotides to non-binding nucleotides, relative to the single-stranded DNA (mt-probe strand) of the gene having a gene modification (mutation gene), of from 10:1 to 1:10.

55. The method as recited in claim 39, wherein at most 60% of nucleotides forming the detectable hybridization probe (sensor probe) do not interact with/bind to the single-stranded DNA (mt-probe strand) of the gene having the modification (mutation gene), based on the total number of nucleotides of the detectable hybridization probe (sensor probe).

56. The method as recited in claim 39, wherein 1 to 15 nucleotides of the detectable hybridization probe (sensor probe) are not capable of interacting with/binding to the single-stranded DNA (mt-probe strand) of the gene having a gene modification (mutation gene).

57. The method as recited in claim 39, wherein a heat-induced detachment of the detectable hybridization probe (sensor probe) from the single-stranded DNA of the mutation gene (mt-probe strand) takes place at a lower temperature than that of the corresponding single-stranded DNA of the wild type gene (wt-probe strand).

58. The method as recited in claim 39, wherein the detectable hybridization probe (sensor probe) emits at least one of a detectable signal, a measurable signal, and a fluorescence signal when interacting with/binding to at least one of the single-stranded DNA of the mutation gene (mt-probe strand), and the corresponding single-stranded DNA of the wild type gene (wt-probe strand).

59. The method as recited in claim 39, wherein the detectable hybridization probe (sensor probe) does not emit a detectable/measurable signal or emits a reduced detectable/measurable signal upon a heat-induced dissociation from at least one of the single-stranded DNA of the mutation gene (mt-probe strand) and the corresponding single-stranded DNA of the wild type gene (wt-probe strand).

60. The method as recited in claim 37, further comprising
providing at least one second hybridization probe (anchor probe),
wherein, at least one of:
1) the at least one second hybridization probe (anchor probe) is different from the detectable hybridization probe (sensor probe),
2) the detectable hybridization probe (sensor probe) and the at least one second hybridization probe (anchor probe) can form a FRET pair; and
3) the at least one second hybridization probe (anchor probe) can bind to the same single-stranded DNA as the detectable hybridization probe (sensor probe).

61. The method as recited in claim 37,
wherein the at least one of a tumor and a cancer is a lung carcinoma such as a non-small cell lung cancer (NSCLC), a small cell lung cancer (SCLC), and a non-small cell lung cancer (NSCLC), and
wherein the protein is at least one of a human protein, a protein which at least one of regulates and induces a cell growth/proliferation, a transmembrane receptor for a growth factor such as with an intrinsic tyrosine kinase activity, and an epidermal growth factor receptor (EGF receptor).

62. The method as recited in claim 37, wherein the at least one gene modification is localized in exon 18, in exon 19, in exon 20 or in exon 21 of an epidermal growth factor receptor (EGF receptor).

63. The method as recited in claim 37, wherein the at least one gene modification is at least one of a deletion in exon 19, a deletion in a region of an amino acid position 746, a deletion in a region of an amino acid position 747, and a deletion in a region of an amino acid position 746 and 747, of the epidermal growth factor receptor (EGF receptor).

64. The method as recited in claim 37, wherein the at least one gene modification is a deletion selected from the group of deletions ΔE746-A750, ΔE746-T751, ΔE746-A750 (ins RP), ΔE746-T751 (ins A/I), ΔE746-T751 (ins VA), ΔE746-S752 (ins A/V), ΔL747-E749 (A750P), ΔL747-A750 (ins P), ΔL747-T751, ΔL747-T751 (ins P/S), ΔL747-S752, ΔL747-S752 (E746V), ΔL747-S752 (P746V), ΔL747-S752 (ins Q), ΔL747-P753, ΔL747-P753 (ins S) and ΔS752-I759.

65. The method as recited in claim 37, wherein the at least one of a tumor and a cancer is at least one of associated with a gene modification such as a translocation in a gene coding for a fusion protein EML4-ALK and is a gene modification such as a translocation in a gene coding for a fusion protein EML4-ALK.

66. The method as recited in claim 37, wherein the at least one of a tumor and a cancer is at least one of:
1) a leukemia such as an acute myeloid leukemia (AML),
2) associated with a gene modification such as an insertion in a gene coding for a receptor tyrosine kinase FLT3, and
3) a gene modification such as an insertion in a gene coding for a receptor tyrosine kinase FLT3.

67. The method as recited in claim 60, wherein the method is carried out by an asymmetric polymerase chain reaction (PCR) using the at least one of the detectable hybridization probe (sensor probe) and optionally
at least one of at least one second hybridization probe (anchor probe), and at least one wild-type-specific blocking agent which inhibits a binding of the at least one detectable hybridization probe (sensor probe) to the gene not having a gene modification (wild type gene),
so as to at least one of selectively increase and selectively amplify the single-stranded DNA having the gene modification (mt-probe strand) of the gene having the modification (mutation gene) and the single-stranded DNA (wt-probe strand) of the gene not having a gene modification (wild type gene), with which the detectable hybridization probe (sensor probe) can interact with/bind to.

68. The method as recited in claim 67, wherein the method includes at least one of:
1) carrying out the asymmetric polymerase chain reaction (PCR) in the presence of primers, such as primers in the form of oligonucleotides,
2) amplifying a gene segment of the gene having a gene modification,
3) amplifying a gene segment of the gene having a gene modification (mutation gene) which corresponds to a gene segment of the gene not having a gene modification (wild type gene),
4) binding a first primer at least substantially specifically to the single-stranded DNA of the mutation gene (mt-probe strand), with which the detectable hybridization probe (sensor probe) can interact,
5) binding a second primer at least substantially specifically to a single-stranded DNA of the gene having a gene modification (mutation gene) which is complementary to the probe strand (mt-complementary strand), and
6) selecting the first primer and the second primer so that at least one of an amount and a concentration of the first primer is greater than at least one of an amount and a concentration of the second primer so that an amplification of the mt-probe strand versus the mt-complementary strand is at least one of increased and intensified.

69. The method as recited in claim 67, wherein at least one of:
the at least one wild type-specific blocking agent is at least one of an oligonucleotide and a polynucleotide, and
the at least one wild-type-specific blocking agent has at least one of a higher specificity, a higher binding affinity, and a higher selectivity with respect to the single-stranded DNA (wt-probe strand) of the gene not having a gene modification (wild type gene).

70. The method as recited in claim 39, wherein, to detect the at least one gene modification, such as following the polymerase chain reaction, the method further comprises at least one of:
1) recording a melting curve;
2) carrying out a melting curve analysis;
3) detecting at least one of a cleavage and a dehybridization, such as of the detectable hybridization probe (sensor probe) from the respective single-stranded DNA of at least one of the gene having a gene modification (mutation gene) and from the gene not having a gene modification (wild type gene), wherein the detecting can occur photometrically such as by measuring a fluorescence,
4) drawing a conclusion from at least one of a melting point, melting points and melting point ranges of the melting curve on an existence of a mutation; and
5) if the mutation exists, determining a nature of the mutation.

71. A composition for use in the context of an asymmetric polymerase chain reaction (PCR) to detect at least one gene modification such as a mutation in a gene, such as a gene that codes for a protein associated with at least one of a tumor and a cancer, the composition comprising:
a detectable wild type-specific hybridization probe (sensor probe);
a first primer which binds at least substantially specifically to a single-stranded DNA of a mutation gene (mt-probe strand) with which the detectable wild-type-specific hybridization probe (sensor probe) can interact;
a second primer which can interact at least substantially specifically with a single-stranded DNA of a mutation gene complementary to the probe strand (mt-complementary strand); and
a wild-type-specific blocking agent which inhibits a binding of the detectable wild-type-specific hybridization probe (sensor probe) to a wild type gene;
wherein, at least one of a content and an amount of the first primer in the composition is greater than at least one of a content and an amount of the second primer in the composition.

72. A method of using the composition as recited in claim 71 to detect at least one gene modification, such as a mutation in a gene, such as in a gene that codes for a protein associated with at least one of a tumor and a cancer, the method comprising:
1) providing the composition as recited in claim 71; and
2) using the composition to detect at least one gene modification.


SEQUENCE LISTING (6 sequences)

SEQ ID NO: 1 - protein, 1210 aa, Homo sapiens [sequence omitted]

SEQ ID NO: 2 - DNA, 26 bp, Artificial Sequence
  PCR oligonucleotide specific for the corresponding gene of human epidermal growth factor receptor (EGFR)
gtcttccttctctctctgtcataggg

SEQ ID NO: 3 - DNA, 18 bp, Artificial Sequence
  PCR oligonucleotide specific for the corresponding gene of human epidermal growth factor receptor (EGFR)
gggcctgaggttcagagc

SEQ ID NO: 4 - DNA, 21 bp, Artificial Sequence
  Real-time PCR probe specific for a section of the wild-type DNA sequence of the EGFR gene in the region of exon 19 (usable as sensor probe for detecting deletions in the exon 19 region)
taattccttgatagcgacggg

SEQ ID NO: 5 - DNA, 30 bp, Artificial Sequence
  Real-time PCR probe specific for a section of the wild-type DNA sequence of the EGFR gene in the region of exon 19 (usable as anchor probe for detecting deletions in the exon 19 region)
attttaactttctcaccttctgggatccag

SEQ ID NO: 6 - DNA, 13 bp, Artificial Sequence
  Blocking agent according to the locked nucleic acid principle, usable for real-time PCR, specifically for a section on the wild-type EGFR gene
taattccttgata